SEQUENCE LISTING (26 sequences)

SEQ ID NO: 1 - DNA, 14 bp, Unknown
  Synthetic
actcacctcgtttc

SEQ ID NO: 2 - DNA, 20 bp, Unknown
  Synthetic
ccttataagcatgtctcata

SEQ ID NO: 3 - DNA, 22 bp, unknown
  Synthetic
agagacagactcacctcgtttc

SEQ ID NO: 4 - DNA, 27 bp, unknown
  synthetic
gagacagtatgagacatgcttataagg

SEQ ID NO: 5 - DNA, 14 bp, unknown
  synthetic
gccgccgctggggc

SEQ ID NO: 6 - DNA, 20 bp, unknown
  synthetic
cctccccacgctctctagcc

SEQ ID NO: 7 - DNA, 15 bp, unknown
  synthetic
ggccgccgctggggc

SEQ ID NO: 8 - DNA, 24 bp, unknown
  synthetic
acagggctagagagcgtggggagg

SEQ ID NO: 9 - DNA, 14 bp, unknown
  synthetic
ggagcgatcaccat

SEQ ID NO: 10 - DNA, 20 bp, unknown
  synthetic
tagacgttcatggtacatac

SEQ ID NO: 11 - DNA, 18 bp, unknown
  synthetic
acagggagcgatcaccat

SEQ ID NO: 12 - DNA, 24 bp, unknown
  synthetic
acaggtatgtaccatgaacgtcta

SEQ ID NO: 13 - DNA, 14 bp, unknown
  synthetic
cggagtgctgggat

SEQ ID NO: 14 - DNA, 20 bp, unknown
  synthetic
cctttgtggtcatgagtttg

SEQ ID NO: 15 - DNA, 16 bp, unknown
  synthetic
agcggagtgctgggat

SEQ ID NO: 16 - DNA, 22 bp, unknown
  synthetic
agcaaactcatgaccacaaagg

SEQ ID NO: 17 - DNA, 20 bp, unknown
  synthetic
catcaacgttcatgtcgcgc

SEQ ID NO: 18 - DNA, 20 bp, unknown
  synthetic
ccttgggtgctcgcagtaaa

SEQ ID NO: 19 - DNA, 14 bp, unknown
  synthetic
tcgtcggcagcgtc

SEQ ID NO: 20 - DNA, 19 bp, unknown
  synthetic
agatgtgtataagagacag

SEQ ID NO: 21 - DNA, 34 bp, unknown
  synthetic
  misc_feature (1)..(14): n is A or G or C or T
nnnnnnnnnnnnnncatcaacgttcatgtcgcgc

SEQ ID NO: 22 - DNA, 15 bp, unknown
  synthetic
gtctcgtgggctcgg

SEQ ID NO: 23 - DNA, 19 bp, unknown
  synthetic
agatgtgtataagagacag

SEQ ID NO: 24 - DNA, 40 bp, unknown
  synthetic
  misc_feature (1)..(20): n is A or G or T or C
nnnnnnnnnnnnnnnnnnnnccttgggtgctcgcagtaaa

SEQ ID NO: 25 - DNA, 20 bp, unknown
  synthetic
  misc_feature (3)..(20): W is A or T; S is G or C; M is A or C; B is C or G or T; D is A or G or T; H is A or C or T; V is A or C or G; and N is A or C or G or T.
ccwswdhshdbvhdnnnnmm

SEQ ID NO: 26 - DNA, 21 bp, unknown
  synthetic
  misc_feature (3)..(21): W is A or T; S is G or C; M is A or C; B is C or G or T; D is A or G or T; H is A or C or T; V is A or C or G; and N is A or C or G or T.
ccswswhdsdhvbdhnnnnmm

(19) United States
(12) Patent Application Publication
(10) Pub. No.: US 2017/0349925 A1
(43) Pub. Date: Dec. 7, 2017

(54) Methods for Nucleic Acid Assembly

(71) Applicant: Gingko Bioworks, Inc., Boston, MA (US)

(72) Inventors: Joseph Jacobson, Newton, MA (US); Ishtiaq E. Saaem, Cambridge, MA (US)

(21) Appl. No.: 15/521,087

(22) PCT Filed: Oct. 20, 2015
(86) PCT No.: PCT/US15/56433

§ 371 Date: Apr. 21, 2017

Related U.S. Application Data
(60) Provisional application No. 62/066,840, filed on Oct. 21, 2014.
(60) Provisional application No. 62/090,083, filed on Dec. 10, 2014.

(51) Int. Cl. C12P 19/34; C12N 15/10

(52) U.S. Cl. CPC C12P 19/34; C12N 15/1031; C12N 15/1065

(57) ABSTRACT

Methods and compositions relate to the assembly of high fidelity nucleic acids. Specifically, nucleic acid molecules having a desired predetermined sequence can be assembled after failure to assemble in conventional assembly. Aspects of the disclosure relate to methods of assembling a target nucleic acid molecule having a desired or predetermined sequence.

### CROSS-REFERENCE TO RELATED APPLICATIONS

This application claims priority to and the benefit of U.S. Provisional Application Nos. 62/066,840 filed Oct. 21, 2014 and 62/090,083 filed Dec. 10, 2014, each of which applications is incorporated herein by reference in their entirety.

### FIELD

Methods and compositions disclosed herein relate to nucleic acid assembly, and particularly assembly of target sequences that are difficult to assemble using conventional technology.

### BACKGROUND

Recombinant and synthetic nucleic acids have many applications in research, industry, agriculture, and medicine. Recombinant and synthetic nucleic acids can be used to express and obtain large amounts of polypeptides, including enzymes, antibodies, growth factors, receptors, and other polypeptides that may be used for a variety of medical, industrial, or agricultural purposes. Recombinant and synthetic nucleic acids also can be used to produce genetically modified organisms including modified bacteria, yeast, mammals, plants, and other organisms. Genetically modified organisms may be used in research (e.g., as animal models of disease, as tools for understanding biological processes, etc.), in industry (e.g., as host organisms for protein expression, as bioreactors for generating industrial products, as tools for environmental remediation, for isolating or modifying natural compounds with industrial applications, etc.), in agriculture (e.g., modified crops with increased yield or increased resistance to disease or environmental stress, etc.), and for other applications. Recombinant and synthetic nucleic acids also may be used as therapeutic compositions (e.g., for modifying gene expression, for gene therapy, etc.) or as diagnostic tools (e.g., as probes for disease conditions, etc.).

Numerous techniques have been developed for modifying existing nucleic acids (e.g., naturally occurring nucleic acids) to generate recombinant nucleic acids. For example, combinations of nucleic acid amplification, mutagenesis, nuclease digestion, ligation, cloning and other techniques may be used to produce many different recombinant nucleic acids. Chemically synthesized polynucleotides are often used as primers or adaptors for nucleic acid amplification, mutagenesis, and cloning.

Techniques also are being developed for de novo nucleic acid assembly whereby nucleic acids are made (e.g., chemically synthesized) and assembled to produce longer target nucleic acids of interest. For example, different multiplex assembly techniques are being developed for assembling oligonucleotides into larger synthetic nucleic acids that can be used in research, industry, agriculture, and/or medicine. However, one limitation of currently available assembly techniques is the relatively high error rate and failure to assemble certain sequences. As such, high fidelity, low cost assembly methods are needed.

### SUMMARY

Aspects of the disclosure relate to methods of assembling a target nucleic acid molecule having a desired or predetermined sequence. In some embodiments, the method comprises:
(a) providing a plurality of nucleic acid molecules assembled from a pool of oligonucleotides;
(b) detecting a target nucleic acid that is misassembled, wherein the target nucleic acid is designed to be constructed from a set of construction oligonucleotides;
(c) selectively amplifying each of the set of construction oligonucleotides from the pool of oligonucleotides; and
(d) assembling the amplified set of construction oligonucleotides to form the target nucleic acid.

In some embodiments, the misassembled nucleic acid (i) is underrepresented in the plurality of assembled nucleic acid molecules and/or (ii) contains an error. The error can be missing one or more construction oligonucleotide. In certain embodiments, the selectively amplifying comprises PCR amplification using a first primer that is universal to all of the construction oligonucleotides and a second primer that is unique to each construction oligonucleotide. Accordingly, each of the set of construction oligonucleotides may be designed to comprise a universal primer binding site located at a first end that binds the first primer, and a unique primer binding set located at a second end that binds the second primer. In some embodiments, each of the set of construction oligonucleotides may contain nested or serial primer binder sites at one or both ends where one or more outer primers and inner primers can bind. In one example, the construction oligonucleotides each have binding sites for a pair of outer primers and a pair of inner primers. One or both of the pair of outer primers may be universal primers. Alternatively, one or both of the pair of outer primers may be unique primers. In some embodiments, in step (c), each of the set of construction oligonucleotides is individually amplified. The construction oligonucleotides can also be pooled into one or more pools for amplification. In one example, all of the set of construction oligonucleotides are amplified in a single pool. In step (d), in certain embodiments, the amplified set of construction oligonucleotides are assembled via polymerase assembly or ligation. The amplified set of construction oligonucleotides are assembled hierarchically or sequentially or in a one-step reaction. By way of example only, hierarchical assembly of oligonucleotides A, B, C and D may include assembling A+B and C+D first, then A+B+C+D. Sequential assembly may include assembling A+B, then A+B+C, and finally A+B+C+D. One-step assembly combines A, B, C and D in one reaction to result in A+B+C+D.

Another aspect relates to a method for assembling a target nuclei acid from a set of construction oligonucleotides, comprising: uniformly amplifying each of the set of construction oligonucleotides from the pool of oligonucleotides; and assembling the amplified set of construction oligonucleotides to form the target nucleic acid.

In some embodiments, the step of uniformly amplifying comprises using a partial degenerate primer for each of the set of construction oligonucleotides. In the partial degenerate primer, 1, 2, 3, 4, 5, 6, 7, 8, 9 or 10 positions can contain 2 or 3 or 4 possible bases, and the remainder of the partial degenerate primer can be specific for the corresponding construction oligonucleotide.

In another embodiment, the step of uniformly amplifying comprises using a primer having a complement strand and an inhibitor strand, wherein the complement strand is partially complementary to each of the set of construction oligonucleotides and the inhibitor strand is designed to compete with the corresponding construction oligonucleotide for binding of the complement strand. The complement strand can be designed to have a complement region and a kick-off region, wherein the complement region binds the inhibitory strand and the kick-off region binds to the corresponding construction oligonucleotide. The complement and inhibitor strand are further designed such that a first hybridization energy of the kick-off region to the corresponding construction oligonucleotide is substantially matched to a second hybridization energy of complement region to the inhibitor strand.

Another aspect relates to a method for assembling a target nuclei acid, comprising:

(a) providing a plurality of nucleic acid molecules assembled from a pool of oligonucleotides;

(b) detecting a target nucleic acid that is misassembled, wherein the target nucleic acid is designed to be constructed from a subset of construction oligonucleotides out of the pool of oligonucleotides;

(c) selectively amplifying at least one of the subset of construction oligonucleotides; and

(d) assembling the amplified subset of construction oligonucleotides to form the target nucleic acid.

In some embodiments, the misassembled target nucleic acid (i) is underrepresented in the plurality of assembled nucleic acid molecules and/or (ii) contains an error. The error can include missing one or more construction oligonucleotides. In certain embodiments, the selectively amplifying step comprises polymerase based reaction using a first primer that is universal to all of the construction oligonucleotides in the subset and a second primer that is unique to the at least one construction oligonucleotide in the subset. Each of the subset of construction oligonucleotides may be designed to comprise a universal primer binding site located at a first end that binds the first primer, and a unique primer binding site located at a second end that binds the second primer. In step (c), each of the subset of construction oligonucleotides may be, in some embodiments, individually amplified. In certain embodiments, all of construction oligonucleotides in the subset can be amplified in step (c) in a single pool. In step (d) in some embodiments, the amplified subset of construction oligonucleotides can be assembled via polymerase based assembly or ligase based ligation. In certain embodiments, in step (d), the amplified subset of construction oligonucleotides are assembled hierarchically.

A further aspect relates to a method for assembling a target nucleic acid from a subset of construction oligonucleotides out of a pool of oligonucleotides, comprising:

(a) selectively, from the pool of oligonucleotides comprising the subset of construction oligonucleotides, and uniformly amplifying each of the subset of construction oligonucleotides; and

(b) assembling the amplified subset of construction oligonucleotides to form the target nucleic acid.

In some embodiments, the amplifying step comprises using a partial degenerate primer for each of the subset of construction oligonucleotides. In the partial degenerate primer, 1, 2, 3, 4, 5, 6, 7, 8, 9 or 10 positions can each contain 2 or 3 or 4 possible nucleotides, and the remainder of the partial degenerate primer can be specific to each construction oligonucleotide in the subset. In certain embodiments, the amplifying step can comprise using a primer having a complement strand and an inhibitor strand, wherein the complement strand is partially complementary to each of the subset of construction oligonucleotides and the inhibitor strand is designed to compete with the corresponding construction oligonucleotide for binding to the complement strand. The complement strand may be designed to have a complement region and a kick-off region, wherein the complement region binds the inhibitory strand and the kick-off region binds the corresponding construction oligonucleotide. In some embodiments, the complement strand and the inhibitor strand may be designed such that a first hybridization energy of the kick-off region and the corresponding construction oligonucleotide is substantially matched to a second hybridization energy of complement region and the inhibitor strand.

In embodiments, methods of the present disclosure are particularly useful during assembly of multiple target nucleic acids (e.g., multiple genes or fragments thereof) where one or more targets fail to be properly built due to various reasons such as underrepresented construction oligonucleotide(s). The failed built can be detected (e.g., by sequencing), and then “rescued” by the methods disclosed herein. Sometimes this method is referred to as “PCR rescue” or “single oligonucleotide rescue” (SOR) or “discrete amplification rescue” (DARe). This method can be particularly useful in assembly of target nucleic acids that have repeat sequences and/or high GC content.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A illustrates steps I, II, and III of a non-limiting exemplary method of preparative cloning according to some embodiments. FIG. 1B illustrates steps IV and V of a non-limiting exemplary method of preparative cloning according to some embodiments. FIG. 1C illustrates the preparative recovery of correct clones, step VI, of a non-limiting exemplary method of preparative cloning according to some embodiments. Stars denote incorrect or undesired sequence sites.

FIGS. 2A-2C illustrate non-limiting exemplary methods of preparative in vitro cloning sample preparation according to some embodiments.

FIG. 3 illustrates a non-limiting exemplary sample processing from nucleic acid constructs (C2G constructs) to in vitro cloning constructs (IVC constructs).

FIG. 4 illustrates a non-limiting exemplary flow chart for sequencing data analysis.

FIG. 5 illustrates a non-limiting exemplary alternative scheme of plasmid-based barcoding.

FIG. 6 illustrates a non-limiting exemplary parsing and scoring parses.

FIGS. 7A-7B illustrate non-limiting embodiments of the separation of source molecules.

FIG. 8 illustrates a non-limiting exemplary isolation of target nucleic acids using degenerate barcodes.

FIG. 9 illustrates a non-limiting exemplary isolation of nucleic acid clones from a pool of constructs using barcodes.

FIG. 10 illustrates a non-limiting exemplary isolation of nucleic acid clones from a pool of constructs using barcodes.

FIG. 11 illustrates a non-limiting exemplary embodiment of bead-based recovery process.

FIG. 12 illustrates a non-limiting example of in vitro cloning integration with assembly.

FIGS. 13A-13B illustrate non-limiting examples of inverted in vitro cloning.

FIGS. 14A-14D illustrate a method according to a non-limiting embodiment for determining barcode pair information. FIG. 14A illustrates a pathway according to one embodiment by which the barcoded ends of the molecules are brought together by blunt end ligation of the constructs into circles. FIG. 14B illustrates a pathway according to another embodiment by which the barcoded ends of the molecules are brought together by blunt end ligation of the constructs into circles. FIG. 14C illustrates a method according to a non-limiting embodiment of attaching barcodes to the synthesized constructs. FIG. 14D illustrates how parallel sequencing of constructs and the isolated barcode pairs can be used to identify the correct molecule for subsequent capture by amplification. X in a sequence denotes an error in the molecule.

FIG. 15 illustrates a non-limiting embodiment for determining barcode pair information.

FIG. 16 illustrates a non-limiting embodiment for amplifying construction oligonucleotides before assembly.

FIG. 17 illustrates a non-limiting embodiment for amplifying construction oligonucleotides followed by assembly.

### DETAILED DESCRIPTION

In one aspect, the methods and compositions descried herein provide a diagnostic for nucleic acid assembly failure and a path for rescue that failure. Exemplary assembly failures, sometimes referred to as “misassembly” or “misassembled target” or “failed built,” include, without limitation, one or more missing construction oligonucleotides from the assembled product, disruption in assembly where the assembled product contains one or more misplaced construction oligonucleotides (sometimes referred to as “chimera”), complete or partial failure in assembly such that no or little assembled product can be detected, and other error(s) in the sequence of the assembled product that is different than the desired or predetermined sequence (e.g., non-contiguous sequence where the assembled product does not represent the entire contiguous, predetermined sequence; one or more insertions; one or more deletions; one or more point mutations; one or more inversions; or any combination of the foregoing). Once any assembly failure is detected or diagnosed, methods and compositions descried herein can be used to rescue that failure.

Techniques have been developed for de novo nucleic acid assembly whereby nucleic acids are made (e.g., chemically synthesized) and assembled to produce longer target nucleic acids of interest. For example, different multiplex assembly techniques are being developed for assembling oligonucleotides into larger synthetic nucleic acids. One technique is solid support (e.g., chip or microarray) based assembly where construction oligonucleotides synthesized and bound on a solid support can be cleaved or otherwise released from the solid support, and the unbound oligonucleotides can then be optionally amplified (e.g., in a polymerase based reaction) before being assembled into multiple target nucleic acids. However, one limitation of currently available assembly techniques is the relatively high error rate and sometimes the inability to build certain constructs. In some cases, one or more construction oligonucleotides may be underrepresented on the solid support due to, e.g., difficulty or failure in oligonucleotide synthesis on the solid support. If the construction oligonucleotides are amplified before assembly, one or more construction oligonucleotides may have, e.g., repeat sequences or high GC content that may result in difficulty or failure in amplification and therefore, may be underrepresented. When one or more construction oligonucleotides are underrepresented, the corresponding target nucleic acid(s) originally designed to contain such underrepresented construction oligonucleotides will be underrepresented as well or will be more likely to have assembly errors (e.g., missing a portion). On the other hand, underrepresented construction oligonucleotides render the other construction nucleotides to present at relatively high level, which may result in additional assembly errors such as the presence of an extraneous sequence or insertion in the assembled product. Additionally assembly errors include cases where during the assembly process when there is some (e.g., partial) complementarity between the sticky ends of two construction oligonucleotides or two subconstructs, they may be joined together to form a misassembled product that does not represent the contiguous, predetermined target sequence. Thus, there is a need to detect, from a pool of assembled nucleic acids constructs, the misassembled constructs having errors or failures, and then rebuild such misassembled constructs.

More particularly, oligonucleotides can be synthesized on a chip, optionally PCR (polymerase chain reaction) amplified using, e.g., universal and/or specific primers, and assembled into a plurality of target nucleic acids. The target nucleic acids can also be PCR amplified before further processing such as sequencing and/or in vitro cloning. Certain aspects of this method are described in U.S. Publication No. US2014/0141982 by Jacobson et al., which is incorporated herein by reference in its entirety. However, it has been observed that certain targets are difficult to build and as a result, are underrepresented or missing, or contain errors (e.g., one or more oligonucleotide sequences are missing). While without wishing to be bound by theory, it is hypothesized that the inability to build a construct may be due to two primary reasons: (1) eschewed ratio of the oligonucleotides required to assemble a construct, and/or (2) that the construct is difficult to amplify via PCR (e.g., due to high GC content and/or repeat sequences). It is further hypothesized that the cause behind eschewed ratio of the oligonucleotides may also be PCR-related, as the result of inherently difficult PCR template oligonucleotides or eschewed representation of a template oligonucleotide within a synthesized chip. It is also hypothesized that these PCR issues are exacerbated when amplifying a mixed pool of templates. Therefore, needs exist to address these issues.

In one aspect of the present disclosure, to re-assemble a misassembled nucleic acid, a discrete or unique primer can be assigned to a construction oligonucleotide and a single-oligo PCR (e.g., using one unique primer, or two unique primers at either end, or nested primers including at least one unique primer and/or at least one universal primer) can be performed from, e.g., a chip or bead, thereby allowing for the highest chance of successful PCR of that construction oligonucleotide. In some embodiments, all construction oligonucleotides which together form a target nucleic acid are amplified. In certain embodiments, a subset of the construction oligonucleotides that are underrepresented are amplified. The construction oligonucleotides may be individually amplified, or be pooled together for amplification, using one or more of universal primers, nested primers, partial degenerative primers, and/or energy balanced primers as disclosed herein. In one example, all construction oligonucleotides are amplified in a single reaction volume or pot, with a subset of construction oligonucleotides (e.g., those previously failed to build and currently being “rescued”, or those predicted, e.g., by sequence analysis, to be difficult to amplify) being amplified with their corresponding unique primers (or a combination of universal primer and unique primer in nested PCR), while the other construction oligonucleotides being amplified via universal primers. In such a single pot reaction, the unique primer sequences can be designed to be specific to the intended subset of construction oligonucleotides and to avoid hybridizing or annealing with sequence segments of the other construction oligonucleotides. The amplified construction oligonucleotides are then assembled to obtain the correct target nucleic acid by, e.g., ligation or polymerase assisted assembly. The assembled target may be subject to sequencing, further PCR amplification, and/or assembly into a vector. In some instances where the target is difficult to PCR amplify, assembly into a vector can enable in vivo amplification.

In some embodiments, the construction oligonucleotides may comprise universal (common to all oligonucleotides), semi-universal (common to at least a portion of the oligonucleotides) or individual or unique primer (specific to each oligonucleotide) binding sites at either the 5′ end or the 3′ end, or both ends. As used herein, the term “universal” primer or primer binding site means that a sequence used to amplify the oligonucleotide is common to all oligonucleotides in a library which can contain 2 or more members. In certain embodiments, each of the construction oligonucleotides in the library can contain a unique primer binding site at either the 5′ end or the 3′ end, or both. In some embodiments, each construction oligonucleotide contains both universal and unique primer binding sites, which can optionally be used together or sequentially in PCR (e.g., in nested PCR). The universal primer binding site may be upstream (5′) or outer to the unique primer biding site. Alternatively, the universal primer binding site may be downstream (3′) or inner to the unique primer biding site. As used herein, the term “nested PCR” refers to polymerase chain reaction involving two sets of primers (three primers P1, P2 and P3 where P1+P2 is a first set and P1+P3 is a second set; or four primers P1, P2, P3 and P4 where P1+P2 is a first set and P3+P4 is a second set), used in two successive runs of or a single-pot of polymerase chain reaction, the second set being designed to amplify a secondary target within the first run product. The corresponding primers used in nested PCR are referred to as “nested primers.”

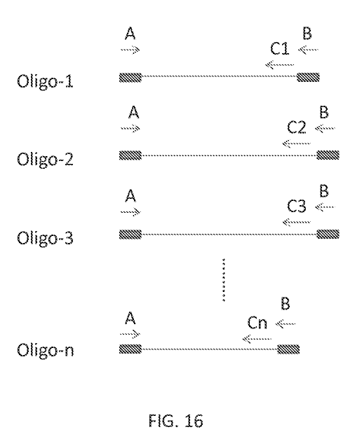

In one embodiment as shown in FIG. 16, a pool of oligonucleotides can be designed with universal primers A and optionally B, which together serve to amplify the entire pool. A nested third primer C1, C2, C3 . . . that is unique to each oligo in the pool can be introduced (also referred to as “nested primers”). Thereby oligo-1 can be amplified with primers A+C1, oligo-2 can be amplified with primers A+C2, oligo-3 can be amplified with primers A+C3 and so on. Subsequently, in a digestion reaction, these orthogonal primers can be removed with the aid of, e.g., a restriction enzyme, thereby leaving the construction oligonucleotides having uniquely complementary cohesive ends between one another such that when assembled, they form the intended target. Other DNA cleaving or gene editing methods can also be used, such as those described in PCT International Application No. PCT/US2015/039517 filed Jul. 8, 2015, incorporated herein by reference in its entirety. A new gene editing tool was recently described in Zetsche et al., Cpfl Is a Single RNA-Guided Endonuclease of a Class 2 CRISPR-Cas System, Cell (2015), www.dx.doi.org/10.1016/j.cell.2015.09.038, incorporated herein by reference in its entirety. Alternatively, C1, C2, C3 . . . may be designed to anneal to sequences that are part of the intended target, thereby eliminating the need to remove the corresponding primer binding sites.

As an alternative, or in addition, to amplifying select construction oligonucleotides using semi-universal or unique primers, such construction oligonucleotides may be provided from an external source. This strategy may be desirable when particular construction oligonucleotides are difficult to amplify. The construction oligonucleotides may be individually provided by direct synthesis or from a commercial source. If two or more construction oligonucleotides happen to be next to one another in the predetermined target sequence, the two or more construction oligonucleotides can be provided in the form of a contiguous fragment that is, e.g., synthesized or pre-assembled.

In certain embodiments, construction oligonucleotides to be assembled into one or more products (e.g., a synthetic gene or cDNA) may be pooled together in a library. A chip can be designed to hold one or more such libraries. Alternatively, beads can be used as a solid support for the oligonucleotides. Before assembly, the oligonucleotides can optionally be amplified via, e.g., PCR to obtain more copies thereof. If there is a sufficient amount of oligonucleotides then amplification is not necessary. Where amplification is performed, in some examples, it may be desirable to produce a more uniform representation of the amplified oligonucleotides. One exemplary method is to control the number of PCR primers (e.g., n) available in the reaction for each oligonucleotide, such that only n more amplified oligonucleotides are produced. This can be achieved by using a highly diluted primer solution having a finite number of primers. Alternatively or additionally, the oligonucleotides and/or their corresponding PCR primers can be pre-designed. In some embodiments, the PCR primers can be designed to contain a partial degenerate sequence where a portion thereof (e.g., 1, 2, 3, 4, 5, 6, 7, 8, 9 or 10, or more positions) contain a number of possible bases (e.g., 2 or 3 or 4), while the remainder of the primer is specific, in each case, to the target oligonucleotide (also referred to as “partial degenerative primers”). This way, only a small percentage of the primers have a total sequence that is specific to the target oligonucleotide. By way of example only, a library may contain 10 different oligonucleotides, and a primer pair can be designed for each library oligonucleotide in the form of XN, where X represents the specific portion (e.g., 10-20 bases long, or shorter or longer) that is specific to a target oligonucleotide and N represents the degenerate portion (e.g., 3-8 bases long, or shorter or longer) in which each position can contain any one of the 4 bases (A, T, C, G). Thus, for each primer that has, for example, a 5-base-long N portion, there are there are 2̂4*2̂4*2̂4*2̂4*2̂4 possible sequences and only 1 of which is specific to its target oligonucleotide. In a PCR reaction solution where a primer is typically provided at 100-500 nM, only a small fraction (1 out of 2̂4*2̂4*2̂4*2̂4*2̂4 under stringent condition; higher if less stringent) anneals to the target oligonucleotide and amplifies therefrom, resulting in a finite number of amplified oligonucleotides.

In some embodiments, the primers are designed so that the standard free energy for hybridization between the specific target oligonucleotide and the primer is close to zero, while the energy for hybridization between an unintended target (even one differing from the actual target by as little as a single nucleotide) and the primer is high enough to make their binding unfavorable by comparison. Such energy landscape distinguishing can be accomplished by designing primers with a complement strand and an inhibitor strand (also referred to as “energy balanced primers”). The complement strand is partially complementary to the oligonucleotide of interest and is designed to have a complement region and a kick-off region. The inhibitor strand is designed to not hybridize to the target and instead compete with the target (or spurious target) for binding of the complement region. The kick-off region is present in the complementary strand, is complementary to a target sequence and is not complementary to an inhibitor region. The hybridization energy of the kick-off region to target is matched or nearly matched to the hybridization energy of complement balance region to inhibitor balance region on the inhibitor strand (adjusting for various other thermodynamic considerations). The sequence of the balance region is rationally designed to achieve this matching under desired conditions of temperature and primer concentration. As a result, the equilibrium for the actual target and primer rapidly approaches 50%.

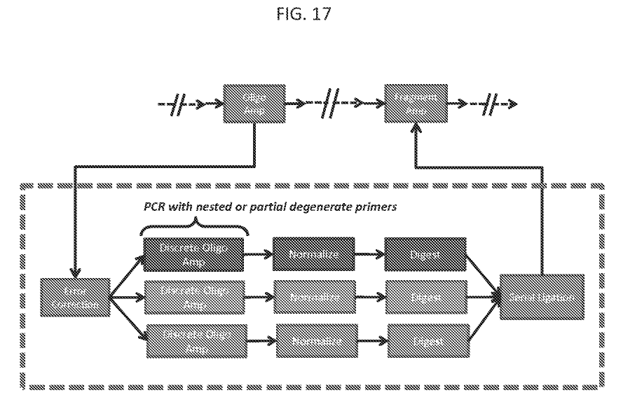

Oligonucleotides can be subject to one or more rounds of amplification, each optionally followed by error correction to remove or correct those contain an error. Each round of amplification can use a universal primer that targets all oligonucleotides, or a nested primer or partial degenerate primer or energy balanced primer as discussed above. In one example as shown in FIG. 17, the first round of amplification uses a universal primer, followed by error correction, a second round of amplification using nested primer or partial degenerate primer, and then digestion and ligation (e.g., serial or hierarchical). It should be noted that the second round of amplification can be done individually for each discrete oligonucleotide to be amplified, or in a combined pool containing two or more oligonucleotides and their corresponding primers. The ligated fragment can be further amplified, error corrected, etc.

One or more of the primers disclosed herein can be methylated such that the amplified product can be digested with a methylation-sensitive nuclease such as MspJI, SgeI and FspEI. Such nuclease shares both type IIM and type IIS properties; thus, it only recognizes the methylation-specific 4-bp sites, ^{m}CNNR (N=A or T or C or G; R=A or G), and cuts DNA outside of this recognition sequences. Methylated primers and use thereof are disclosed in Chen et al., Nucleic Acids Research, 2013, Vol. 41, No. 8, e93, which is incorporated herein by reference in its entirety.

Amplification of the construction oligonucleotides before assembly is optional. In some embodiments, all of the construction oligonucleotides can be assembled together without amplification (e.g., when the construction oligonucleotides are provided at relatively uniform and sufficient amount). In certain embodiments, only a subset of the construction oligonucleotides (e.g., those that are underrepresented) are amplified before assembly. Assembly can be done in one step, or hierarchically in more than one step, or sequentially by adding one construction oligonucleotide at a time, or any combination of the foregoing where some construction oligonucleotides are assembled using one method while others are assembled using another method. In one example, the construction oligonucleotides may, without amplification, be first assembled into two or more subconstructs. The subconstructs can be optionally amplified and then assembled, in one or more steps, into the final construct having the predetermined target sequence.

In some embodiments, hairpin-based assembly strategy as described in U.S. Publication No. 2008/0287320, incorporated herein by reference in its entirety, can be used to assembly the predetermined target sequence or a portion thereof. Hairpins can also be used in error removal. For example, one or more construction oligonucleotides can be designed as a single-stranded nucleic acid having both the plus (sense) strand and the minus (antisense) strand connected by a loop element, such that the plus strand and minus strand can form a self-complementary stem and loop structure. Where there is error (e.g., during oligonucleotide synthesis) on either the plus strand or minus strand, or both strands, a mismatch is formed in the stem and loop structure which can be detected by a mismatch recognition enzyme such as MutS. The description in U.S. Publication No. 2008/0287320 regarding hairpin, stem and loop structure and mismatch recognition enzyme, in particular paragraphs [0091]-[0119], is incorporated by reference in its entirety.

Aspects of the disclosure can be used to isolate nucleic acid molecules from large numbers of nucleic acid fragments efficiently, and/or to reduce the number of steps required to generate large nucleic acid products, while reducing error rate. Aspects of the disclosure can be incorporated into nucleic assembly procedures to increase assembly fidelity, throughput and/or efficiency, decrease cost, and/or reduce assembly time. In some embodiments, aspects of the disclosure may be automated and/or implemented in a high throughput assembly context to facilitate parallel production of many different target nucleic acid products. In some embodiments, nucleic acid constructs may be assembled using starting nucleic acids obtained from one or more different sources (e.g., synthetic or natural polynucleotides, nucleic acid amplification products, nucleic acid degradation products, oligonucleotides, etc.). Aspects of the disclosure relate to the use of a high throughput platform for sequencing nucleic acids such as assembled nucleic acid constructs to identify high fidelity nucleic acids at lower cost. Such platform has the advantage to be scalable, to allow multiplexed processing, to allow for the generation of a large number of sequence reads, to have a fast turnaround time and to be cost efficient.

Some aspects the disclosure relate to the preparation of construction oligonucleotides for high fidelity nucleic acid assembly. Aspects of the disclosure may be useful to increase the throughput rate of a nucleic acid assembly procedure and/or reduce the number of steps or amounts of reagent used to generate a correctly assembled nucleic acid. In certain embodiments, aspects of the disclosure may be useful in the context of automated nucleic acid assembly to reduce the time, number of steps, amount of reagents, and other factors required for the assembly of each correct nucleic acid. Accordingly, these and other aspects of the disclosure may be useful to reduce the cost and time of one or more nucleic acid assembly procedures.

The methods described herein may be used with any nucleic acid molecules, library of nucleic acids or pool of nucleic acids. For example, the methods of the disclosure can be used to generate nucleic acid constructs, oligonucleotides or libraries of nucleic acids having a predefined sequence. In some embodiments, the nucleic acid library may be obtained from a commercial source or may be designed and/or synthesized onto a solid support (e.g. array).

### Parsing

In some embodiments, a nucleic acid sequence of interest (e.g., requiring assembly) can be parsed into a set of construction oligonucleotides that together comprise the nucleic acid sequence of interest. For example, in a first step, sequence information can be obtained. The sequence information may be the sequence of a nucleic acid of interest that is to be assembled. Such sequence is generally referred to herein as “predetermined sequence.” In some embodiments, the predetermined sequence may be received in the form of a purchase order from a customer. One of ordinary skill in the art, e.g., a molecular biologist, will appreciate that the exact sequence is irrelevant and that any and all sequences (i.e., of any length or size or nucleic acid sequence) can be constructed and/or rescued by one or more methods disclosed herein.

In some embodiments, the sequence may be received as a nucleic acid sequence (e.g., DNA or RNA). In some embodiments, the sequence may be received as a protein sequence. The sequence may be converted into a DNA sequence. For example, if the sequence obtained is an RNA sequence, the Us may be replaced with Ts to obtain the corresponding DNA sequence. If the sequence obtained is a protein sequence, it may be converted into a DNA sequence using appropriate codons for the amino acids.

In some embodiments, the sequence information may be analyzed to determine an assembly strategy, according to one or more of the following: the number of the junctions, the length of the junctions, the sequence of the junctions, the number of the fragments, the length of the fragments, the sequence of the fragments to be assembled by cohesive end ligation, to generate the predefined nucleic acid sequences of interest. In some embodiments, the fragments can be assembled by cohesive end ligation or by polymerase chain assembly.

In some embodiments, the assembly design is based on the length of the construction oligonucleotides and/or the number of junctions. For example, according to some embodiments, the length of the fragments can have an average length range of 98 to 104 bps (base pairs) or 89 to 104 bps or 50 to 200 bps or 100 to 146 bps or about 142 bps. In some embodiments, the design that results in the smaller number of fragments or junctions can be selected.

In some embodiments, the sequence analysis may involve scanning the junctions and selecting junctions having one or more of the following feature(s): each junction is 4 or more nucleotides long, each junction differs from the other junctions by at least 2 nucleotides, and/or each junction differs from the other junctions by one or more nucleotide in the last 3 nucleotides of the junction sequence. Junction can then be scored according to the junction distance (also referred herein as Levenshtein distance) in the junction sequences. As used herein, the junction distance or Levenshtein distance corresponds to the measure of the difference between two sequences. Accordingly, the junction distance or Levenshtein distance between a first and a second junction sequences corresponds to the number of single nucleotide changes required to change the first sequence into the second sequence. For example, a 1 nucleotide difference in a sequence of 4 nucleotides corresponds to a junction distance of 1, a 2 nucleotides difference in a sequence of 4 nucleotides corresponds to a junction distance of 2. Junction distances can be averaged. In some embodiments, the junctions are designed so as to have an average of 2 or higher junction distance. In some embodiments, the design that results in the greater junction distance can be selected.

In some embodiments, all possible parses which satisfy the predetermined constraints are analyzed. If no valid parses are found, constraints can be relaxed to find a set of possible oligonucleotide sequences and junctions. For example, the constraint on the length of oligonucleotides can be relaxed to include oligonucleotides having shorter or longer lengths.

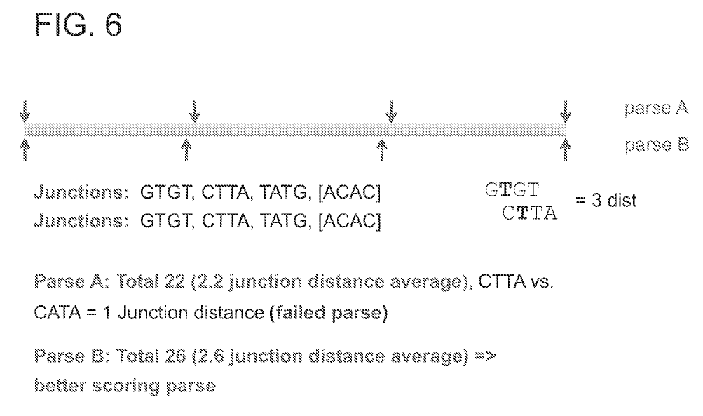

In some embodiments, all possible parses which satisfy the predetermined constraints are ranked based on any metric provided herein. For example, each parse can be ranked based on the average junction distance metric (as illustrated in FIG. 6), the GC content, the complexity of the oligonucleotide sequence, and/or any other suitable metric.

In some embodiments, the sequence analysis may involve scanning for the presence of one or more interfering sequence features that are known or predicted to interfere with oligonucleotide synthesis, amplification or assembly. For example, an interfering sequence structure may be a sequence that has a low GC content (e.g., less than 30% GC, less than 20% GC, less than 10% GC, etc.) over a length of at least 10 bases (e.g., 10-20 bases, 20-50 bases, 50-100 bases, or more than 100 bases), or sequence that may be forming secondary structures or stem-loop structures.

In some embodiments, after the construct qualification and parsing steps, synthetic construction oligonucleotides for the assembly may be designed (e.g. sequence, size, and number). Synthetic oligonucleotides can be generated using standard DNA synthesis chemistry (e.g. phosphoramidite method). Synthetic oligonucleotides may be synthesized on a solid support, such as for example a microarray, using any appropriate technique known in the art. Oligonucleotides can be eluted from the microarray prior to be subjected to amplification or can be amplified on the microarray.

As used herein, an oligonucleotide may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 300 nucleotides long (e.g., from about 30 to 250, from about 40 to 220 nucleotides long, from about 50 to 200 nucleotides long, from about 60 to 180 nucleotides long, or from about 65 or about 150 nucleotides long), between about 100 and about 200 nucleotides long, between about 200 and about 300 nucleotides long, between about 300 and about 400 nucleotides long, or between about 400 and about 500 nucleotides long. However, shorter or longer oligonucleotides may be used. An oligonucleotide may be a single-stranded or double-stranded nucleic acid. As used herein the terms “nucleic acid”, “polynucleotide”, “oligonucleotide” are used interchangeably and refer to naturally-occurring or non-naturally occurring, synthetic polymeric forms of nucleotides. In general, the term “nucleic acid” includes both “polynucleotide” and “oligonucleotide” where “polynucleotide” may refer to longer nucleic acid (e.g., more than 1,000 bases or base pairs, more than 5,000 bases or base pairs, more than 10,000 bases or base pairs, etc.) and “oligonucleotide” may refer to shorter nucleic acid (e.g., 10-500 bases or base pairs, 20-400 bases or base pairs, 40-200 bases or base pairs, 50-100 bases or base pairs, etc.). The nucleic acid molecules of the present disclosure may be formed from naturally occurring nucleotides, for example forming deoxyribonucleic acid (DNA) or ribonucleic acid (RNA) molecules. Alternatively, naturally-occurring nucleic acids may include structural modifications to alter their properties, such as in peptide nucleic acids (PNA) or in locked nucleic acids (LNA). The solid phase synthesis of nucleic acid molecules with naturally occurring or artificial bases is well known in the art. The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. In some embodiments, the sequence of the nucleic acids does not exist in nature (e.g., a cDNA or complementary DNA sequence, or an artificially designed sequence).

As used herein, the term monomer refers to a member of a set of small molecules which are and can be joined together to form an oligomer, a polymer or a compound composed of two or more members. The particular ordering of monomers within a polymer is referred to herein as the “sequence” of the polymer. The set of monomers includes but is not limited to example, the set of common L-amino acids, the set of D-amino acids, the set of synthetic and/or natural amino acids, the set of nucleotides and the set of pentoses and hexoses. Aspects of the disclosure described herein primarily with regard to the preparation of oligonucleotides, but could readily be applied in the preparation of other polymers such as peptides or polypeptides, polysaccharides, phospholipids, heteropolymers, polyesters, polycarbonates, polyureas, polyamides, polyethyleneimines, polyarylene sulfides, polysiloxanes, polyimides, polyacetates, or any other polymers.

Usually nucleosides are linked by phosphodiester bonds. Whenever a nucleic acid is represented by a sequence of letters, it will be understood that the nucleosides are in the 5′ to 3′ order from left to right. In accordance to the IUPAC notation, “A” denotes deoxyadenosine, “C” denotes deoxycytidine, “G” denotes deoxyguanosine, “T” denotes deoxythymidine, “U” denotes the ribonucleoside, uridine. In addition, there are also letters which are used when more than one kind of nucleotide could occur at that position: “W” (i.e. weak bonds) represents A or T, “S” (strong bonds) represents G or C, “M” (for amino) represents A or C, “K” (for keto) represents G or T, “R” (for purine) represents A or G, “Y” (for pyrimidine) represents C or T, “B” represents C, G or T, “D” represents A, G or T, “H” represents A, C or T, “V” represents A, C, or G and “N” represents any base A, C, G or T (U). It is understood that nucleic acid sequences are not limited to the four natural deoxynucleotides but can also comprise ribonucleoside and non-natural nucleotides.

In some embodiments, the methods and compositions provided herein can use oligonucleotides that are immobilized on a surface or substrate (e.g., support-bound oligonucleotides) where either the 3′ or 5′ end of the oligonucleotide is bound to the surface. Support-bound oligonucleotides comprise for example, oligonucleotides complementary to construction oligonucleotides, anchor oligonucleotides and/or spacer oligonucleotides. As used herein the term “support”, “substrate” and “surface” are used interchangeably and refers to a porous or non-porous solvent insoluble material on which polymers such as nucleic acids are synthesized or immobilized. As used herein “porous” means that the material contains pores having substantially uniform diameters (for example in the nm range). Porous materials include paper, synthetic filters, polymeric matrices, etc. In such porous materials, the reaction may take place within the pores or matrix. The support can have any one of a number of shapes, such as pin, strip, plate, disk, rod, bends, cylindrical structure, particle, including bead, nanoparticles and the like. The support can have variable widths. The support can be hydrophilic or capable of being rendered hydrophilic. The support can include inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass, magnetic controlled pore glass, ceramics, metals, and the like etc.; either used by themselves or in conjunction with other materials. In some embodiments, oligonucleotides are synthesized on an array format. For example, single-stranded oligonucleotides are synthesized in situ on a common support wherein each oligonucleotide is synthesized on a separate or discrete feature (or spot) on the substrate. In some embodiments, single-stranded oligonucleotides can be bound to the surface of the support or feature. As used herein the term “array” refers to an arrangement of discrete features for storing, amplifying and releasing oligonucleotides or complementary oligonucleotides for further reactions. In some embodiments, the support or array is addressable: the support includes two or more discrete addressable features at a particular predetermined location (i.e., an “address”) on the support. Therefore, each oligonucleotide molecule of the array is localized to a known and defined location on the support. The sequence of each oligonucleotide can be determined from its position on the support.

In some embodiments, oligonucleotides are attached, spotted, immobilized, surface-bound, supported or synthesized on the discrete features of the surface or array. Oligonucleotides may be covalently attached to the surface or deposited on the surface. Arrays may be constructed, custom ordered or purchased from a commercial vendor (e.g., Agilent, Affymetrix, Nimblegen). Various methods of construction are well known in the art e.g., maskless array synthesizers, light directed methods utilizing masks, flow channel methods, spotting methods, etc. In some embodiments, construction and/or selection oligonucleotides may be synthesized on a solid support using maskless array synthesizer (MAS). Maskless array synthesizers are described, for example, in PCT Application No. WO 99/42813 and in corresponding U.S. Pat. No. 6,375,903. Other examples are known of maskless instruments which can fabricate a custom DNA microarray in which each of the features in the array has a single-stranded DNA molecule of desired sequence. Other methods for synthesizing oligonucleotides include, for example, light-directed methods utilizing masks, flow channel methods, spotting methods, pin-based methods, and methods utilizing multiple supports. Light directed methods utilizing masks (e.g., VLSIPS™ methods) for the synthesis of oligonucleotides is described, for example, in U.S. Pat. Nos. 5,143,854, 5,510,270 and 5,527,681. These methods involve activating predefined regions of a solid support and then contacting the support with a preselected monomer solution. Selected regions can be activated by irradiation with a light source through a mask much in the manner of photolithography techniques used in integrated circuit fabrication. Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. This process can also be effected through the use of a photoresist which is compatible with the growing surface bound molecules and synthesis chemistries involved. Other steps, such as washing unreacted monomer solution from the support, can be optionally used. Other applicable methods include mechanical techniques such as those described in U.S. Pat. No. 5,384,261. Additional methods applicable to synthesis of oligonucleotides on a single support are described, for example, in U.S. Pat. No. 5,384,261. For example, reagents may be delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites. Flow channel methods involve, for example, microfluidic systems to control synthesis of oligonucleotides on a solid support. For example, diverse polymer sequences may be synthesized at selected regions of a solid support by forming flow channels on a surface of the support through which appropriate reagents flow or in which appropriate reagents are placed. Spotting methods for preparation of oligonucleotides on a solid support involve delivering reactants in relatively small quantities by directly depositing them in selected regions. In some steps, the entire support surface can be sprayed or otherwise coated with a solution, if it is more efficient to do so. Precisely measured aliquots of monomer solutions may be deposited dropwise by a dispenser that moves from region to region. Pin-based methods for synthesis of oligonucleotides on a solid support are described, for example, in U.S. Pat. No. 5,288,514. Pin-based methods utilize a support having a plurality of pins or other extensions. The pins are each inserted simultaneously into individual reagent containers in a tray. An array of 96 pins is commonly utilized with a 96-container tray, such as a 96-well microtiter dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously.

In another embodiment, a plurality of oligonucleotides may be synthesized or immobilized on multiple supports. One example is a bead-based synthesis method which is described, for example, in U.S. Pat. Nos. 5,770,358; 5,639,603; and 5,541,061. For the synthesis of molecules such as oligonucleotides on beads, a large plurality of beads is suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

Pre-synthesized oligonucleotide and/or polynucleotide sequences may be attached to a support or synthesized in situ using light-directed methods, flow channel and spotting methods, inkjet methods, pin-based methods and bead-based methods set forth in the following references: McGall et al. (1996) Proc. Natl. Acad. Sci. U.S.A. 93:13555; Synthetic DNA Arrays In Genetic Engineering, Vol. 20:111, Plenum Press (1998); Duggan et al. (1999) Nat. Genet. S21:10; Microarrays: Making Them and Using Them In Microarray Bioinformatics, Cambridge University Press, 2003; U.S. Patent Application Publication Nos. 2003/0068633 and 2002/0081582; U.S. Pat. Nos. 6,833,450, 6,830,890, 6,824,866, 6,800,439, 6,375,903 and 5,700,637; and PCT Publication Nos. WO 04/031399, WO 04/031351, WO 04/029586, WO 03/100012, WO 03/066212, WO 03/065038, WO 03/064699, WO 03/064027, WO 03/064026, WO 03/046223, WO 03/040410 and WO 02/24597; the disclosures of which are incorporated herein by reference in their entirety for all purposes. In some embodiments, pre-synthesized oligonucleotides are attached to a support or are synthesized using a spotting methodology wherein monomers solutions are deposited dropwise by a dispenser that moves from region to region (e.g., ink jet). In some embodiments, oligonucleotides are spotted on a support using, for example, a mechanical wave actuated dispenser.

In some embodiments, each nucleic acid fragment or construct (also referred herein as nucleic acid of interest) being assembled may be between about 100 nucleotides long and about 1,000 nucleotides long (e.g., about 200, about 300, about 400, about 500, about 600, about 700, about 800, about 900). However, longer (e.g., about 2,500 or more nucleotides long, about 5,000 or more nucleotides long, about 7,500 or more nucleotides long, about 10,000 or more nucleotides long, etc.) or shorter nucleic acid fragments may be assembled using an assembly technique (e.g., shotgun assembly into a plasmid vector). It should be appreciated that the size of each nucleic acid fragment may be independent of the size of other nucleic acid fragments added to an assembly. However, in some embodiments, each nucleic acid fragment may be approximately the same size.

Aspects of the disclosure relate to methods and compositions for the production and/or selective isolation of nucleic acid constructs having a predetermined sequence of interest. As used herein, the term “predetermined sequence” means that the sequence of the target polymer (e.g., DNA) is known and chosen before synthesis or assembly of the polymer. In some embodiments, starting from a predetermined sequence, an assembly strategy and associated construction oligomers that together comprise the target polymer can be devised, using, for example, a computer algorithm.

In particular, aspects of the disclosure is described herein primarily with regard to the preparation of nucleic acids molecules, the sequence of the oligonucleotide or polynucleotide being known and chosen before the synthesis or assembly of the nucleic acid molecules. In some embodiments of the technology provided herein, immobilized oligonucleotides or polynucleotides are used as a source of material. In various embodiments, the methods described herein use pluralities of construction oligonucleotides, each oligonucleotide having a target sequence being determined based on the sequence of the final nucleic acid constructs to be synthesized (also referred herein as nucleic acid of interest). In one embodiment, oligonucleotides are short nucleic acid molecules. For example, oligonucleotides may be from 10 to about 300 nucleotides, from 20 to about 400 nucleotides, from 30 to about 500 nucleotides, from 40 to about 600 nucleotides, or more than about 600 nucleotides long. However, shorter or longer oligonucleotides may be used. Oligonucleotides may be designed to have different length. In some embodiments, the sequence of the polynucleotide construct may be divided up into a plurality of shorter sequences (e.g. construction oligonucleotides) that can be synthesized in parallel and assembled into a single or a plurality of desired polynucleotide constructs using the methods described herein. Nucleic acids, such as construction oligonucleotides, may be pooled from one or more arrays to form a library or pool of nucleic acids before being processed (e.g. tagged, diluted, amplified, sequenced, isolated, assembled etc.).

According to some aspects of the disclosure, each nucleic acid sequence to be assembled (also referred herein as nucleic acid source molecules) can comprise an internal predetermined target sequence having a 5′ end and a 3′ end and additional flanking sequences at the 5′ end and/or at the 3′ end of the internal target sequence. In some embodiments, the internal target sequences or nucleic acids including the internal target sequences and the additional 5′ and 3′ flanking sequences can be synthesized onto a solid support as described herein.

In some embodiments, the synthetic nucleic acid sequences comprise an internal target sequence, and non-target sequences upstream and downstream the target sequence. In some embodiments, the non-target sequences can include a sequence ID (SeqID) at the 3′ end (downstream) and the 5′ end (upstream) of the target sequence for identification of similar target sequences and a sequencing handle (H) at the 3′ end and the 5′ end of the target sequence for mutiplexed sample preparation. The sequencing handle can be at the 3′ end and 5′ end of the sequence ID. In some embodiments, the sequence ID is 10 nucleotides in length. In some embodiments, the sequencing handle H is 20 nucleotides in length. However shorter and longer sequence ID and/or sequencing handles can be used. In some embodiments, the nucleic acid sequences can be synthesized with additional sequences, such as oligonucleotide tag sequences. For example, the nucleic acid sequences can be designed so that they include an oligonucleotide tag sequence chosen from a library of oligonucleotide tag sequences, as described herein. In some embodiments, the nucleic acid sequences can be designed to have an oligonucleotide tag sequence including a sequence common across a set of nucleic acid constructs. The term “common sequence” means that the sequences are identical. In some embodiments, the common sequences can be universal sequences. Yet in other embodiments, the 5′ oligonucleotide tag sequences are designed to have common sequences at their 3′ end and the 3′ oligonucleotide tag sequences are designed to have common sequences at their 5′ end. For example, the nucleic acid can be designed to have a common sequence at the 3′ end of the 5′ oligonucleotide tag and at the 5′ end of the 3′ oligonucleotide tag. The library of oligonucleotide tag sequences can be used for nucleic acid construct to be assembled from a single array. Yet in other embodiments, the library of oligonucleotide tags can be reused for different constructs produced from different arrays. In some embodiments, the library of oligonucleotide tag sequences can be designed to be universal. In some embodiments, the nucleic acid or the oligonucleotide tags are designed to have additional sequences. The additional sequences can comprise any nucleotide sequence suitable for nucleic acid sequencing, amplification, isolation or assembly in a pool.

### Preparative In Vitro Cloning (IVC)

Provided herein are preparative in vitro cloning methods or strategies for de novo high fidelity nucleic acid synthesis. In some embodiments, the in vitro cloning methods can use oligonucleotide tags. Yet in other embodiments, the in vitro cloning methods do not necessitate the use of oligonucleotide tags.

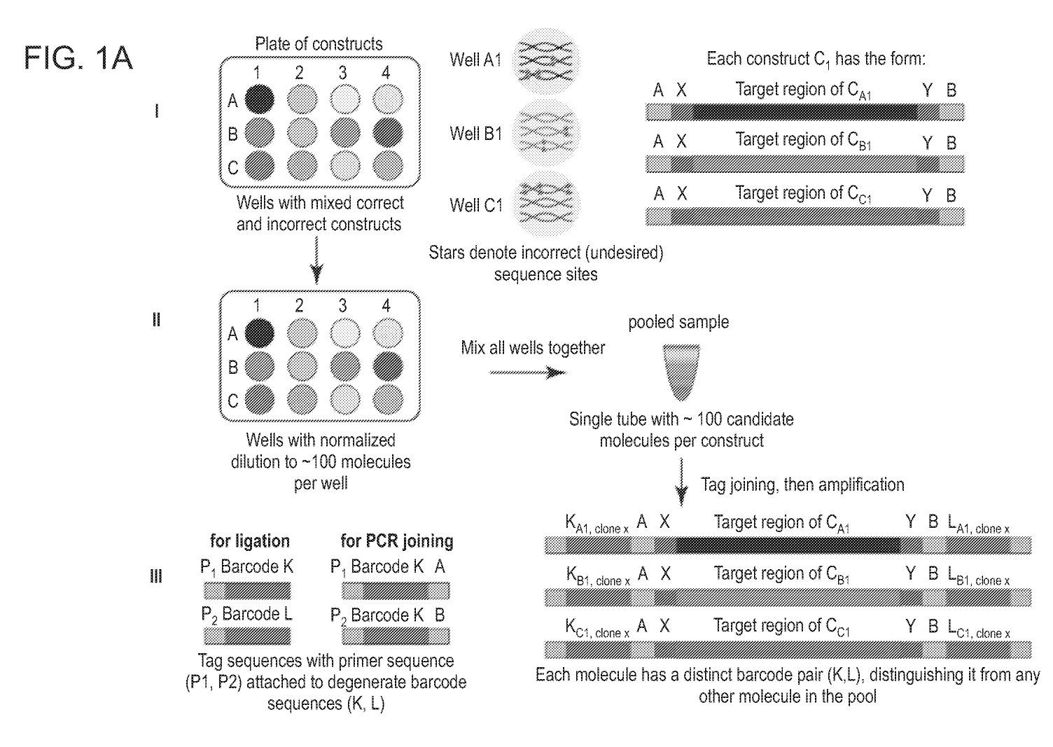

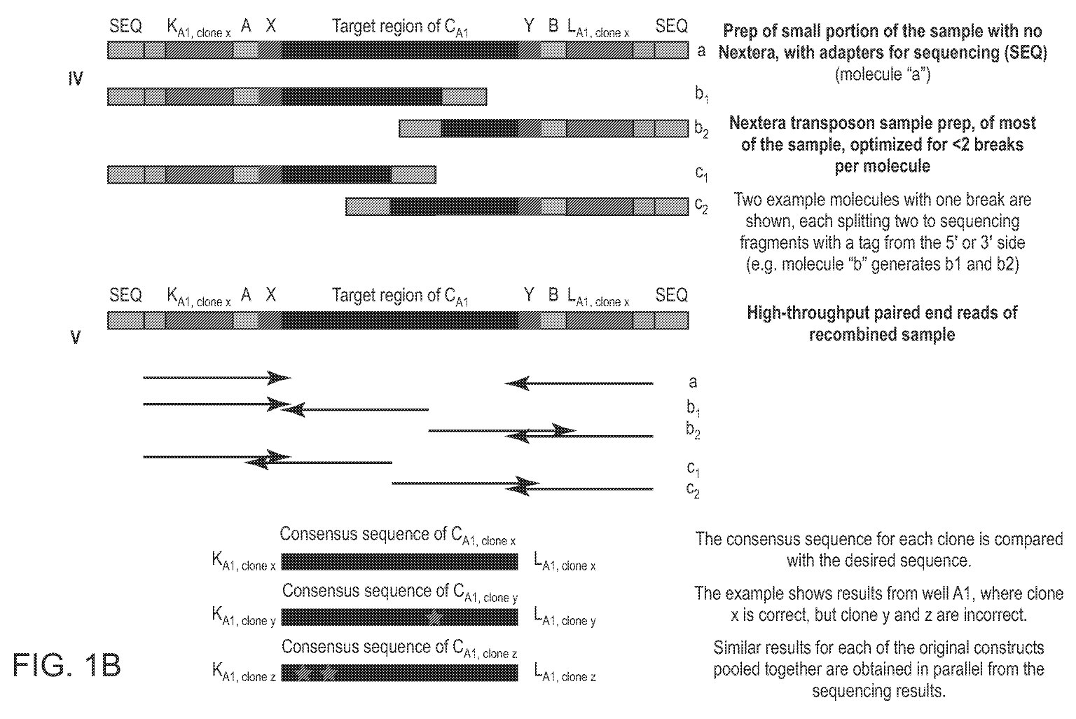

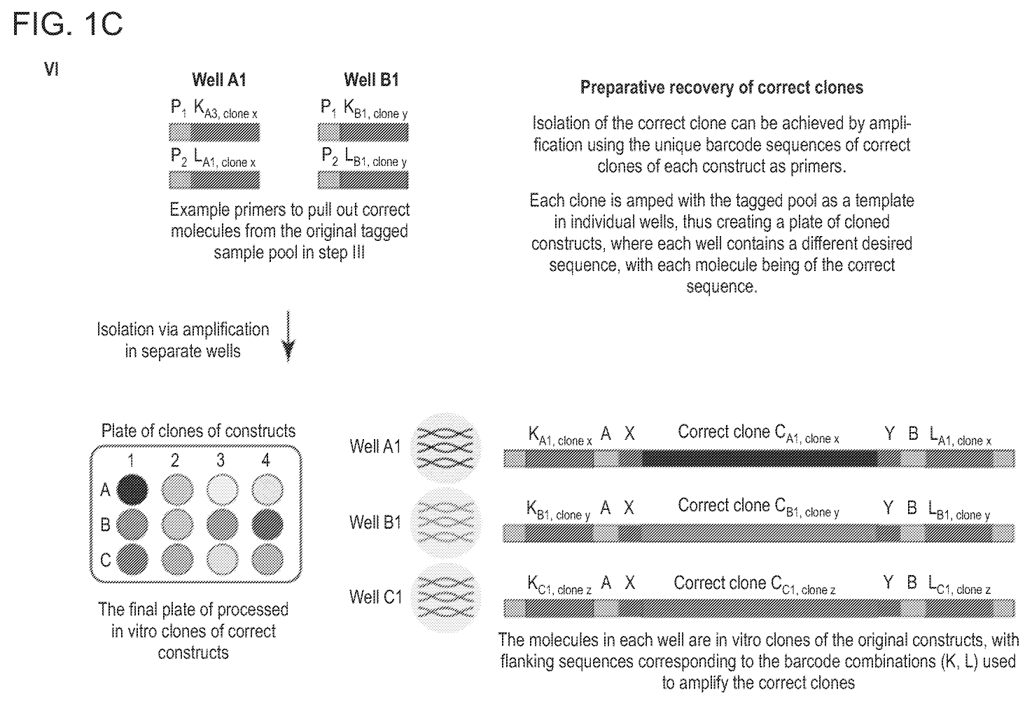

In some embodiments, the methods described herein allow for the cloning of nucleic acid sequences having a desired or predetermined sequence from a pool of nucleic acid molecules. In some embodiments, the methods may include analyzing the sequence of target nucleic acids for parallel preparative cloning of a plurality of target nucleic acids. For example, the methods described herein can include a quality control step and/or quality control readout to identify the nucleic acid molecules having the correct sequence. FIGS. 1A-1C show an exemplary method for isolating and cloning nucleic acid molecules having predetermined sequences. In some embodiments, the nucleic acid can be first synthesized or assembled onto a support. For example, the nucleic acid molecules can be assembled in a 96-well plate with one construct per well. In some embodiments, each nucleic acid construct (C₁ through C_{N}, FIGS. 1A-1C) has a different nucleotide sequence. For example, the nucleic acid constructs can be non-homologous nucleic acid sequences or nucleic acid sequences having a certain degree of homology. Yet in other embodiments, a plurality of nucleic acid molecules having a predefined sequence, e.g. C₁ through C_{N}, can be deposited at different location or well of a solid support. In some embodiments, the limit of the length of the nucleic acid constructs can depend on the efficiency of sequencing the 5′ end and the 3′ end of the full length target nucleic acids via high-throughput paired end sequencing. One skilled in the art will appreciate that the methods described herein can bypass the need for cloning via the transformation of cells with nucleic acid constructs in propagatable vectors (i.e. in vivo cloning). In addition, the methods described herein eliminate the need to amplify candidate constructs separately before identifying the target nucleic acids having the desired sequences.

One skilled in the art would appreciate that after oligonucleotide assembly, the assembly product may contain a pool of sequences containing correct and incorrect assembly products. For example, referring to FIG. 1A, each well of the plate (nucleic acid construct C₁ through C_{N}) can be a mixture of nucleic acid molecules having correct or incorrect sequences (incorrect sequence sites being represented by a star). The errors may result from sequence errors introduced during the oligonucleotide synthesis, or during the assembly of oligonucleotides into longer nucleic acids. In some instances, up to 90% of the nucleic acid sequences may be unwanted sequences. Devices and methods to selectively isolate the correct nucleic acid sequence from the incorrect nucleic acid sequences are provided herein. The correct sequence may be isolated by selectively isolating the correct sequence(s) from the other incorrect sequences as by selectively moving or transferring the desired assembled polynucleotide of predefined sequence to a different feature of the support, or to another plate. Alternatively, polynucleotides having an incorrect sequence can be selectively removed from the feature comprising the polynucleotide of interest. According to some methods of the disclosure, the assembly nucleic acid molecules may first be diluted within the solid support in order to obtain a normalized population of nucleic acid molecules. As used herein, the term “normalized” or “normalized pool” means a nucleic acid pool that has been manipulated, to reduce the relative variation in abundance among member nucleic acid molecules in the pool to a range of no greater than about 1000-fold, no greater than about 100-fold, no greater than about 10-fold, no greater than about 5-fold, no greater than about 4-fold, no greater than about 3-fold or no greater than about 2-fold. In some embodiments, the nucleic acid molecules are normalized by dilution. For example, the nucleic acid molecules can be normalized such as the number of nucleic acid molecules is in the order of about 5, about 10, about 20, about 30, about 40, about 50, about 60, about 60, about 70, about 80, about 90, about 100, about 1000 or higher. In some embodiments, each population of nucleic acid molecules can be normalized by limiting dilution before pooling the nucleic acid molecules to reduce the complexity of the pool. In some embodiments, to ensure that at least one copy of the target nucleic acid sequence is present in the pool, dilution is limited to provide for more than one nucleic acid molecule. In some embodiments, the oligonucleotides can be diluted serially. In some embodiments, the device (for example, an array or microwell plate, such as 96 wells plate) can integrate a serial dilution function. In some embodiments, the assembly product can be serially diluted to a produce a normalized population of nucleic acids. The concentration and the number of molecules can be assessed prior to the dilution step and a dilution ratio is calculated in order to produce a normalized population. In an exemplary embodiment, the assembly product is diluted by a factor of at least 2, at least 3, at least 4, at least 5, at least 6, at least 7, at least 10, at least 20, at least 50, at least 100, at least 1,000 etc. . . . . In some embodiments, prior to sequencing, the target nucleic acid sequences can be diluted and placed for example, in distinct wells or at distinct locations of a solid support or on distinct supports.

In some embodiments, the normalized populations of nucleic acid molecules can be pooled to create a pool of nucleic acid molecules having different predefined sequences. In some embodiments, each nucleic acid molecule in the pool can be at a relatively low complexity. Yet in other embodiments, normalization of the nucleic acid molecules can be performed after mixing the different population of nucleic acid molecules present at high concentration.

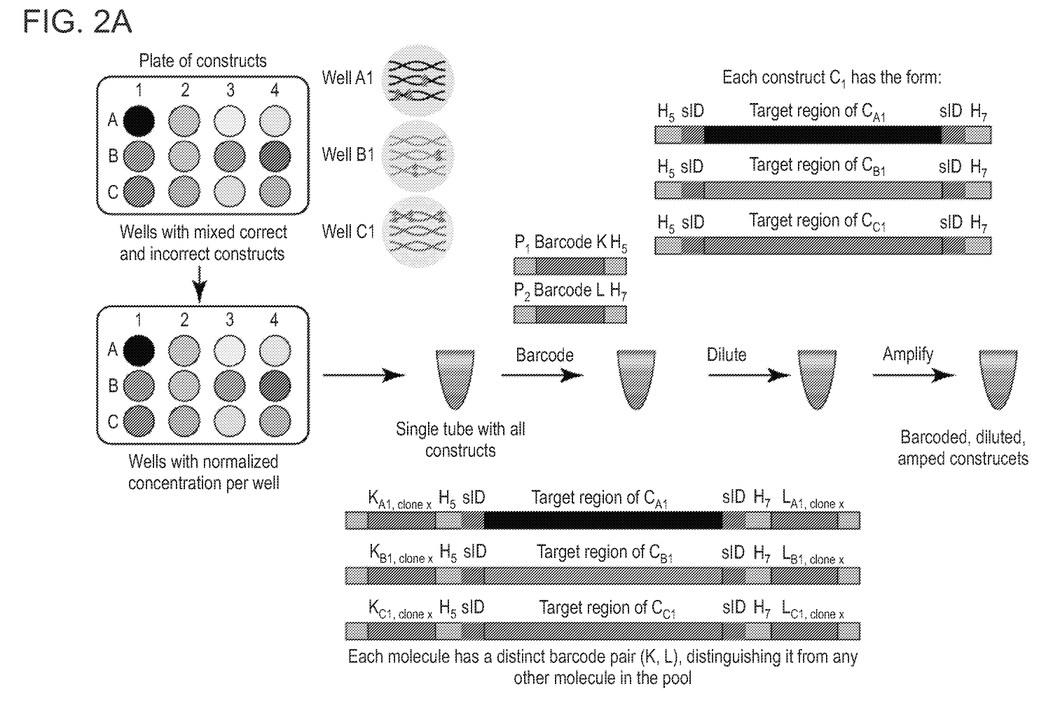

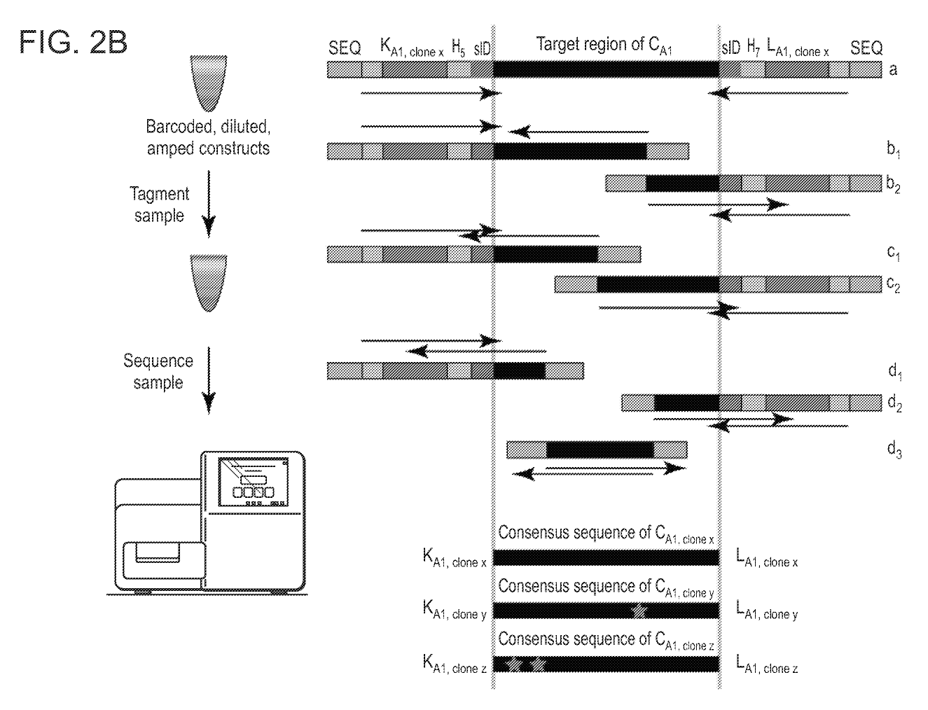

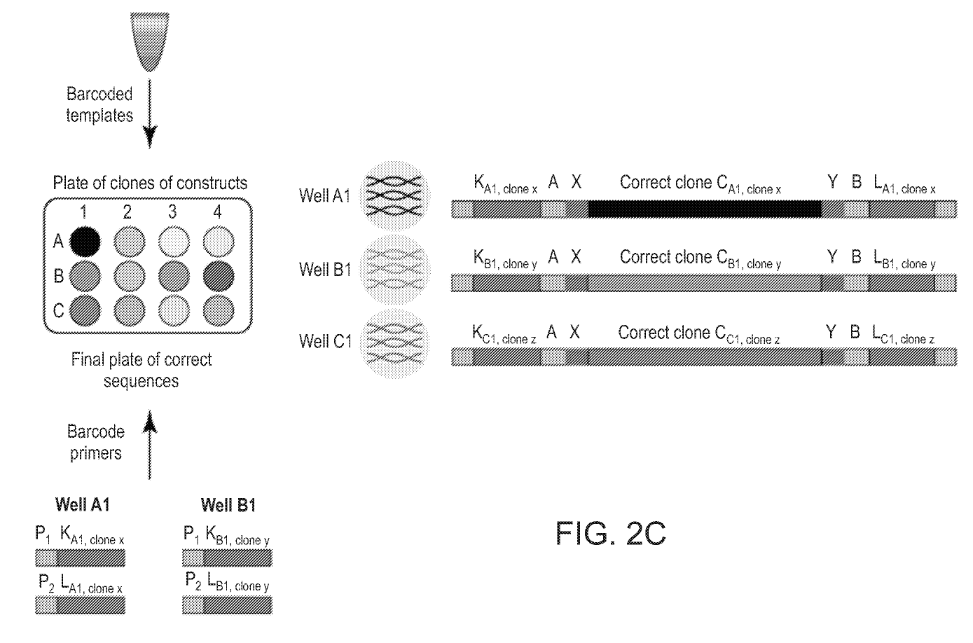

Yet in other embodiments, the methods of the disclosure comprise the following steps as illustrated in FIG. 2A: (a) providing a pool of different nucleic acid constructs (also referred herein as source molecules); (b) providing a repertoire of oligonucleotide tags, each oligonucleotide tag comprising a unique nucleotide tag sequence or barcode; (c) attaching at the 5′ end and at the 3′ end an oligonucleotide tag (K and L) to each source molecule in the pool of nucleic acid molecules, such that substantially all different molecules in the pool have a different oligonucleotide tag pair (K, L) attached thereto and so as to associate a barcode to a specific source molecule, and (d) diluting the tagged nucleic acid sequences; (e) obtaining a paired end read for each nucleic acid molecule; and (f) sorting the nucleic acid molecules having the desired predetermine sequence according to the identity of the barcodes. As used herein, the term “barcode” refers to a unique oligonucleotide tag sequence that allows a corresponding nucleic acid sequence to be identified. By designing the repertoire or library of barcodes to form a library of barcodes large enough relative to the number of nucleic acid molecules, each different nucleic acid molecule can have a unique barcode pair. In some embodiments, the library of barcodes comprises a plurality of 5′ end barcodes and a plurality of 3′ end barcodes. Each 5′ end barcode of the library can be design to have 3′ end or internal sequence common to each member of the library. Each 3′ end barcode of the library can be design to have 5′ end or internal sequence common to each member of the library

In some embodiments, the methods further comprise digesting the tagged source molecules using Nextera™ tagmentation and sequencing using MiSeq®, HiSeq® or higher throughput next generation sequencing platforms. The Nextera™ tagmented paired reads generally generate one sequence with an oligonucleotide tag sequence for identification, and another sequence internal to the construct target region (as illustrated in FIG. 2C). With high throughput sequencing, enough coverage can be generated to reconstruct the consensus sequence of each tag pair construct and determine if the sequence is correct (i.e. error-free sequence).

In some embodiments, the nucleic acid molecules can be pooled from one or more solid supports for multiplex processing. The nucleic acid molecules can be diluted to keep a tractable number of clones per target nucleic acid molecule. Each nucleic acid molecule can be tagged by adding a unique barcode or pair of unique barcodes to each end of the molecule. Diluting the nucleic acid molecules prior to attaching the oligonucleotide tags can allow for a reduction of the complexity of the pool of nucleic acid molecules thereby enabling the use of a library of barcodes of reduced complexity. The tagged molecules can then be amplified. In some embodiments, the oligonucleotide tag sequence can comprise a primer binding site for amplification (FIG. 2C). In some embodiments, the oligonucleotide tag sequence can be used as a primer-binding site. Amplified tagged molecules can be subjected to tagmentation and subjected to paired-read sequencing to associate barcodes with the desired target sequence. The barcodes can be used as primers to recover the sequence clones having the desired sequence. Amplification methods are well known in the art. Examples of enzymes with polymerase activity which can be used for amplification by PCR are NA polymerase (Klenow fragment, T4 DNA polymerase), heat stable DNA polymerases from a variety of thermostable bacteria (Taq, VENT, Pfu or Tfl DNA polymerases) as well as their genetically modified derivatives (TaqGold, VENTexo, Pfu exo), or KOD Hifi DNA polymerases. In some embodiments, amplification by chimeric PCR can reduce signal to noise of barcode association.

In other embodiments, the nucleic acid molecules can be pooled from one or more array for multiplex processing. As described herein, the nucleic acid molecules can be designed to include a barcode at the 5′ and at the 3′ ends. In some embodiments, the barcodes can have common sequences within and across a set of constructs. For example, the barcodes can be universal for each construct assembled from a single array. In some embodiments, the barcodes can have common junction sequences or common primer binding site sequences.

In some embodiments, barcodes can be added to the nucleic acid molecules and tagged nucleic acid molecules can be diluted before being subjected to amplification. Amplified tagged molecules can be subjected to tagmentation and sequenced to associate the barcode pairs to each nucleic acid molecule. In some embodiments, one read of each read pair is used for sequencing barcoded end. The read pairs without any barcodes can be filtered out. Sequencing error rate can be removed by consensus calling. Nucleic acid molecules having the desired sequence can be isolated for example using the barcodes as primers.

According to some methods of the disclosure, the nucleic acid sequences (construction oligonucleotides, assembly intermediates or assembled nucleic acid of interest) may first be diluted in order to obtain a clonal population of target polynucleotides (i.e. a population containing a single target polynucleotide sequence). As used herein, a “clonal nucleic acid” or “clonal population” or “clonal polynucleotide” are used interchangeably and refer to a clonal molecular population of nucleic acids, i.e. to nucleic acids that are substantially or completely identical to each other. Accordingly, the dilution based protocol provides a population of nucleic acid molecules being substantially identical or identical to each other. In some embodiments, the polynucleotides can be diluted serially. The concentration and the number of molecules can be assessed prior to the dilution step and a dilution ratio can be calculated in order to produce a clonal population.

In some embodiments, next-generation sequencing (NGS) spot location or microfluidic channel location can act as a nucleic acid construct identifier eliminating the need for designing construct specific barcodes.

In some embodiments, when using NGS with multiple flow cells (e.g. Hiseq® 2000), it is possible to obtain an average of one clone of each gene per flow cell. As determined by the Poisson distribution, limiting dilution should result in a single-hit, e.g. one clone per well. Poisson statistics gives that if the average number of clones of each gene is one per flow cell then approximately ⅓ of the flow cells will have 0 clones, ⅓ will have 1 clone and ⅓ will have 2 clones. Therefore, if the error rate is such that N clones are required in order to yield a perfect or error-free full length construct, then 3*N flow cells would be required to have high likelihood that at least one flow cell will contain a clonal representation of the perfect construct. For example, if N=4, 12 flow cells would be required. In some embodiments, after sequencing the clones inside the flow cell, means can be provided for collecting the effluent of each flow cell into separate wells. Sequencing data can then used to identify the collection wells that contain the nucleic acid(s) having the predetermined sequence. After determination of which nucleic acids having the predetermined sequence are in which collection wells, primers that are specific to the nucleic acids having the predetermined sequences may then be used to amplify nucleic acids having the predetermined sequences from their appropriate well. In such embodiments, primers can be complementary of the nucleic acid sequences of interest and/or oligonucleotide tags.

### Tag Oligonucleotides

In some embodiments, the 5′ end and the 3′ end of each nucleic acid molecules within the pool can be tagged with a pair of tag oligonucleotide sequence. In some embodiments, the tag oligonucleotide sequence can be composed of common DNA primer regions and unique “barcode” regions such as a specific nucleotide sequence. In some embodiments, the number of tag nucleotide sequences can be greater than the number of molecules per construct (i.e. 10-1000 molecules in the dilution).

In some embodiments, the barcode sequence may also act as a primer binding site to amplify the barcoded nucleic acid molecules or to isolate the nucleic acid molecules having the desired predetermined sequence. In such embodiments, the term barcode and oligonucleotide tag can be used interchangeably. In such embodiments, the terms “barcoded nucleic acids” and “tagged nucleic acids” can be used interchangeably. It should be appreciated that the oligonucleotide tags may be of any suitable length and composition. In some embodiments, the oligonucleotide tags can be designed such as (a) to allow generation of a sufficient large repertoire of barcodes to allow each nucleic acid molecule to be tagged with a unique barcode at each end; and (b) to minimize cross hybridization between different barcodes. In some embodiments, the nucleotide sequence of each barcode is sufficiently different from any other barcode of the repertoire so that no member of the barcode repertoire can form a dimer under the reactions conditions, such as the hybridization conditions, used.

In some embodiments, the barcode sequence can be 6 bp, 7 bp, 8 bp, 9 bp, 10 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp or more than 30 bp in length. In some embodiments, the 5′ end barcode sequence and the 3′ end barcode sequence can differ in length. For example, the 5′ barcode can be 14 nucleotides in length and the 3′ barcode can be 20 nucleotides in length. In some embodiments, the length of the barcode can be chosen to minimize reduction in barcode space, maximize barcode space at the 3′ end for primability, allows error correction for barcodes, and/or minimize the variation of barcode melting temperatures. For example, the melting temperatures of the barcodes within a set can be within 10° C. of one another, within 5° C. of one another or within 2° C. of one another.

Each barcode sequence can include a completely degenerate sequence, a partially degenerate sequence or a non-degenerate sequence.

For example, a 6 bp, 7 bp, 8 bp, or longer nucleotide tag can be used. In some embodiments, a degenerate sequence having 8 degenerate bases wherein each position can be any natural or non-natural nucleotide) can be used and generates 65,536 unique barcodes. In some embodiments, the length of the nucleotide tag can be chosen such as to limit the number of pairs of tags that share a common tag sequence for each nucleic acid construct.

One of skill in the art would appreciate that a completely degenerate sequence can give rise to a high number of different barcodes but also to higher variations in primer melting temperature Tm. Melting temperature is the temperature at which a population of double-stranded nucleic acid molecules becomes half dissociated into single-strands. Equations for calculating the Tm of nucleic acids are well known in the art. For example, a simple estimate of the Tm value can be calculated by the equation Tm=81.5±0.41 (% G+C) when the nucleic acid are in aqueous solution at 1M NaCl. In some embodiments, the barcode sequences are coded barcode and may comprise a partially degenerate sequence combined with fixed or constant nucleotides. In some embodiments, the barcodes can include one or more of the following: (a) degenerate bases N at the 3′ end; (b) one or more C at the 5′ end (to restrict the Tm); (c) stretch comprising W, D, H, S, B, V and M.

In some embodiments, the barcodes are coded barcodes and may include, but are not limited to, a library of barcodes having the following sequences:

Barcode 1: CCWSWDHSHDBVHDNNNNMM (SEQ ID NO: 25). This 20 bases barcode has the same barcode degeneracy space than 13N.

Barcode 2: CCSWSWHDSDHVBDMM (SEQ ID NO: 26). This 21 bases barcode has some degenerate bases switched in location as compared to Barcode 1. It should be noted that primers can be distinguished between Barcode 1 and Barcode 2.

In some embodiments, barcodes sequences can be designed, analyzed and ranked to generate a ranked list of nucleotide tags that are enriched for both perfect sequence and primer performance. It should be appreciated that the coded barcodes provide a method for generating primers with tighter Tm range.

In some embodiments, the tag oligonucleotide sequences or barcodes can be joined to each nucleic acid molecule to form a nucleic acid molecule comprising a tag oligonucleotide sequence at its 5′ and 3′ ends. In some embodiments, the tag oligonucleotide sequences or barcodes can be ligated to blunt end nucleic acid molecules using a ligase. For example, the ligase can be a T7 ligase or any other ligase capable of ligating the tag oligonucleotide sequences to the nucleic acid molecules. Ligation can be performed under conditions suitable to avoid concatamerization of the nucleic acid constructs. In other embodiments, the nucleic acid molecules are designed to have at their 5′ and 3′ ends a sequence that is common or complementary to the tag oligonucleotide sequences. In some embodiments, the tag oligonucleotide sequences and the nucleic acid molecules having common sequences can be joined as adaptamers by polymerase chain reaction. As illustrated in FIG. 2A, barcodes can be joined at the 5′ end and the 3′ end of the sequencing handle H (A and B), which are flanking the internal target sequence. In some embodiments, each source molecule synthesized on a first solid support has a common pair of sequencing handles at its 5′ and 3′ end. For example, oligonucleotides synthesized on a first solid support has a first pair of sequencing handles (A1, B1), and oligonucleotides synthesized on a second solid support has a second pair of sequencing handles (A2, B2), etc. . . .

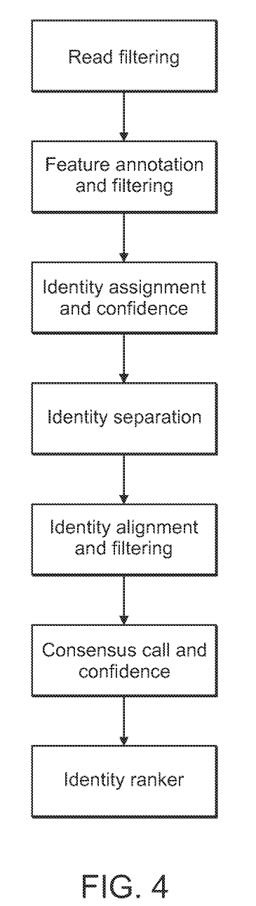

Yet in other embodiments, barcoding can be introduced by ligation to the 5′ end and the 3′ end of a nucleic acid molecule without the addition of sequence identifiers SeqID and/or sequencing handles H. Accordingly, the construct primers are still intact and can act as sequence identifiers. This process can have the advantage to use nucleic acid constructs having an internal target sequence and a primer region at the 5′ end and the 3′ end of the target sequence as synthesized onto an array and to have greater control to normalize the construct. In some embodiments, the barcoding can be introduced using a plasmid-based methodology as illustrated in FIG. 4 comprising the steps of (1) providing a barcoded vector (e.g. pUC19 vector), (2) providing a nucleic assembly construct or oligonucleotide, (3) phosphorylating the nucleic acid constructs; (4) ligating the barcoded vector and the nucleic assembly constructs, and (5) pooling ligation products; and (6) subjecting the ligation products to dilution and/or amplification. For example, the linearized vector comprises 5′ and 3′ flanking regions. In some embodiments, the flanking regions may be designed to have an external barcode and internal sequence adaptors. For example, the flanking regions can comprise a barcode, a tagmentation adaptor and M13 sequences. It should be appreciated that this alternative barcoding scheme is not necessarily plasmid-based and that any linear nucleic acid fragment having a barcode at its 5′ end and 3′ end can be used.

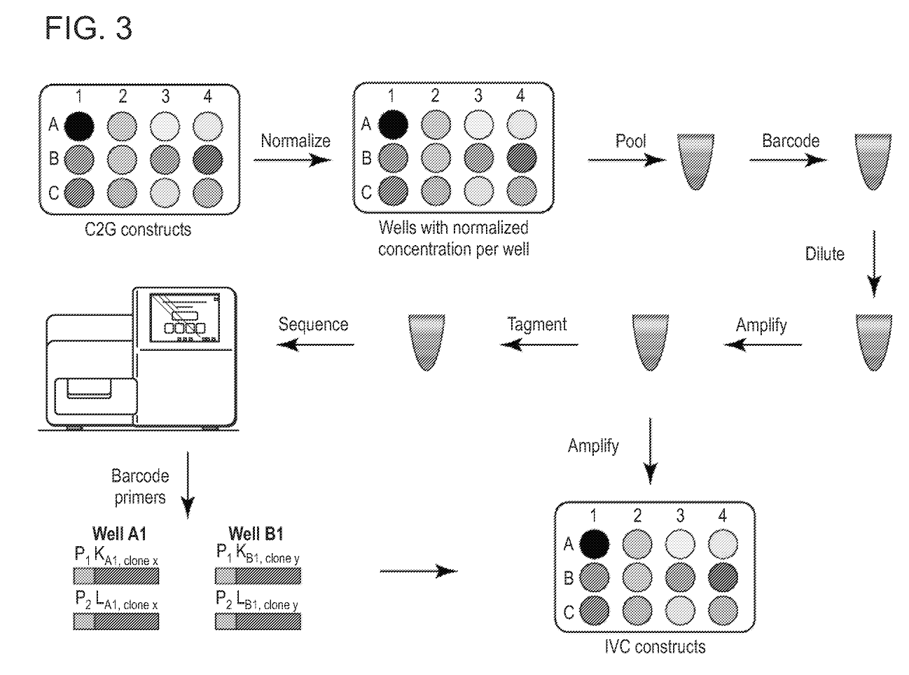

FIG. 3 illustrates the workflow of the foregoing process of tagging a population of target nucleic acid sequences with an oligonucleotide tag, sequencing the molecules to get both the oligonucleotide tag and the internal target sequencing information, and recovering the desired tagged construct sequences. The flow for this workflow could be simplified as:

population of target molecules (A)=>tag (B)=>sequencing (C)=>recover desired target nucleic sequence (D).

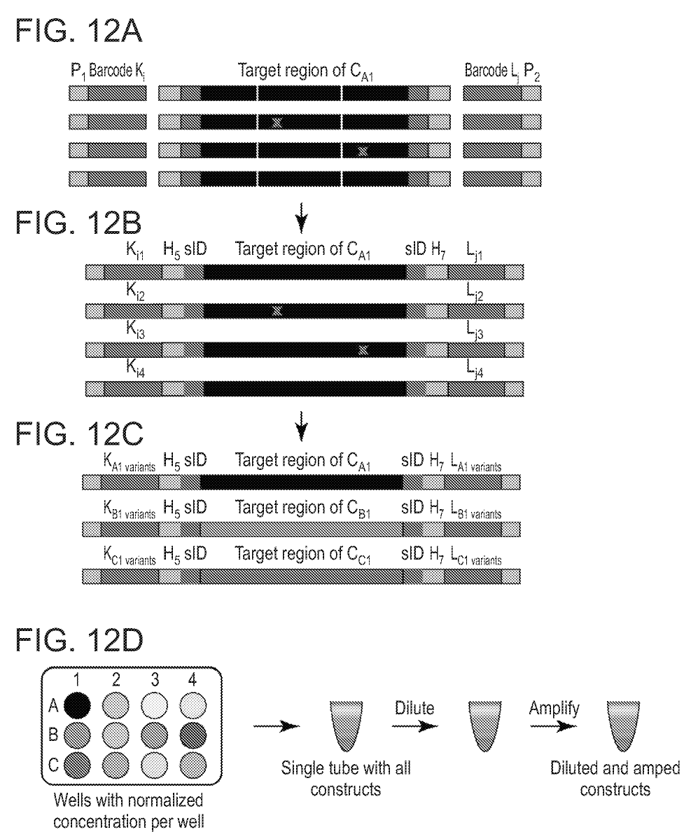

Yet in other embodiments, and referring to FIGS. 12A-B, the nucleic acid constructs can be assembled from a plurality of internal target sequence fragments and unique barcode sequences. The unique barcode sequences can be designed to be assembled at the 5′ end and 3′ end of the internal target sequence simultaneously with the target sequences, to create a population of molecules having unique flanking barcoding sequences and interior target regions of interest. In some embodiments, the 5′ end internal target sequence fragment is designed to have at its 5′ end a sequence identifier SeqID and/or sequencing handle H and the 3′ end internal target sequence fragments is designed to have at its 3′ end a sequence identifies SeqID and/or sequencing handle H. Such process has the advantage to integrate the in vitro cloning process (IVC process) with the assembly process (also referred herein as C2G assembly process). As illustrated in FIGS. 12A-12B, each assembled molecule having the internal target of interest has a distinct pair (Kᵢ,Lᵢ), such as (Kᵢ₁, Lᵢ₂), (Kᵢ₂, Lᵢ₂) etc. . . . of sequences distinguishing it from other molecules in a pool of nucleic acid constructs. In some embodiments, a plurality of constructs having different internal target sequences of interest (for example C_{A1}, C_{B1} and C_{C1}) can be mixed in a pool (FIG. 12C). The different constructs can be diluted, amplified and sequenced as described herein and as illustrated in FIG. 12D. The nucleic acid molecules having the desired sequence can be sorted according to the identity of the corresponding unique pair of barcodes.

One of skill in art will appreciate that the foregoing process has the advantage not to subject the constructs to tagging process, as the core population of molecules is essentially already equivalent to process point B in the workflow above. The workflow could then be described as follow: population of unique target molecules (A′)=>sequencing (C)=>recover desired target nucleic sequence (D).

### Sequencing

In some embodiments, the target nucleic acid sequence or a copy of the target nucleic acid sequence can be isolated from a pool of nucleic acid sequences, some of them containing one or more sequence errors. As used herein, a copy of the target nucleic acid sequence refers to a copy using template dependent process such as PCR. In some embodiments, sequence determination of the target nucleic acid sequences can be performed using sequencing of individual molecules, such as single molecule sequencing, or sequencing of an amplified population of target nucleic acid sequences, such as polony sequencing. In some embodiments, the pool of nucleic acid molecules are subjected to high throughput paired end sequencing reactions, such as using the HiSeq®, MiSeq® (Illumina) or the like or any suitable next-generation sequencing system (NGS).

In some embodiments, the nucleic acid molecules are amplified using the common primer sequences on each tag oligonucleotide sequence. In some embodiments, the primer can be universal primers or unique primer sequences. Amplification allows for the preparation of the target nucleic acids for sequencing, as well as to retrieve the target nucleic acids having the desired sequences after sequencing. In some embodiments, a sample of the nucleic acid molecules is subjected to transposon-mediated fragmentation and adapter ligation to enable rapid preparation for paired end reads using high throughput sequencing systems. For example, the sample can be prepared to undergo Nextera™ tagmentation (Illumina).

One skilled in the art will appreciate that it can be important to control the extent of the fragmentation and the size of the nucleic acid fragments to maximize the number of reads in the sequencing paired reads and thereby allow for sequencing the desired length of the fragment. In some embodiments, the paired end reads can generate one sequence with a tag for identification, and another sequence which is internal to the construct target region. With high throughput sequencing, enough coverage can be generated to reconstruct the consensus sequence of each tag pair construct and determine if the construct sequence is correct. In some embodiments, it is preferable to limit the number of breakage to less than 2, less than 3, or less than 4. In some embodiments the extent of the fragmentation and/or the size of the fragments can be controlled using appropriate reaction conditions such as by using the suitable concentration of transposon enzyme and controlling the temperature and time of incubation. Suitable reaction conditions can be obtained by using known amounts of a test library and titrating the enzyme and time to build a standard curve for actual sample libraries. In some embodiments, a portion of the sample which is not used for fragmentation can be mixed back into the fragmented sample and processed for sequencing.

The sample can then be sequenced on a platform that generates paired end reads. Depending on the size of the individual DNA constructs, the number of constructs mixed together, and the estimated error rate of the populations, the appropriate platform can be chosen to maximize the number of reads desired and minimize the cost per construct.

The sequencing of the nucleic acid molecules results in reads with both of the tags from each molecule in the paired end reads. The paired end reads can be used to identify which pairs of tags were ligated or PCR joined and the identity of the molecule.

### Data Analysis

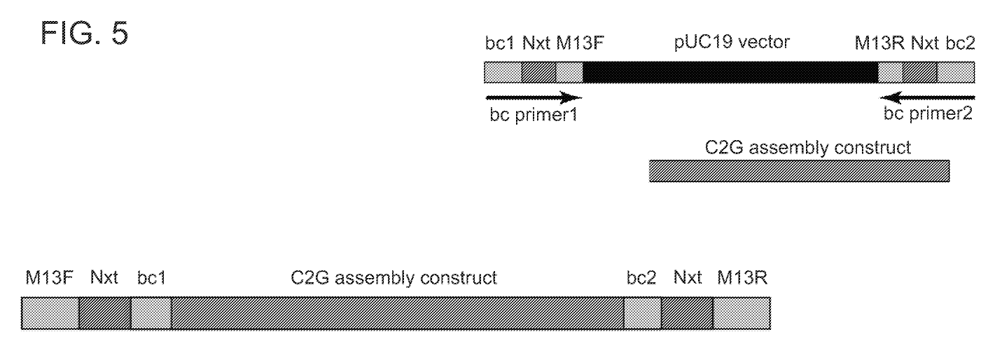

In some embodiments, sequencing data or reads are analyzed according to the scheme of FIG. 5. A read can represent consecutive base calls associated with a sequence of a nucleic acid. It should be understood that a read could include the full length sequence of the sample nucleic acid template or a portion thereof such as the sequence comprising the barcode sequence, the sequence identifier, and a portion of the target sequence. A read can comprise a small number of base calls, such as about eight nucleotides (base calls) but can contain larger numbers of base calls as well, such as 16 or more base calls, 25 or more base calls, 50 or more base calls, 100 or more base calls, or 200 or more nucleotides or base calls.

For data analysis, reads for which one tag is paired with multiple other tags for the same construct are discarded, because this would result in ambiguity as to which clone the data came from.

The sequencing results can then be analyzed to determine the sequences of each clone of each construct. For each paired read where one read contains a tag sequence, the identity of the molecule each sequencing read comes from is known, and the construct sequence itself can be used to distinguish between constructs with the same tag. The other read from the paired read can be used to build a consensus sequence of the internal regions of the molecule. From these results, a mapping of tag pairs corresponding to correct target sequence for each construct can be generated.

According to one embodiment, the analysis can comprise one or more of the following: (1) feature annotation; (2) feature correction; (3) identity assignment and confidence; (4) consensus call and confidence; and (5) preparative isolation.

Aspects of the disclosure provide the ability to generate a consensus sequence for each nucleic acid construct. Each base called in a sequence can be based upon a consensus base call for that particular position based upon multiple reads at that position. These multiple reads are then assembled or compared to provide a consensus determination of a given base at a given position, and as a result, a consensus sequence for the particular sequence construct. It will be appreciated that any method of assigning a consensus determination to a particular base call from multiple reads of that position of sequence, are envisioned and encompassed by the present disclosure. Methods for determining such call are known in the art. Such methods can include heuristic methods for multiple-sequence alignment, optimal methods for multiple sequences alignment, or any methods know in the art. In some embodiments, the sequence reads are aligned to a reference sequence (e.g. predetermined sequence of interest). High throughput sequencing requires efficient algorithms for mapping multiple query sequences such as short reads of the sequence identifiers or barcodes to such reference sequences.

According to some aspects of the disclosure, feature annotation comprises finding primary features and secondary features. For example, using alignment of the two reads of sequence identifiers SeqID in a read pairs allow for filtering constructs that do not have the correct sequence identifiers at the 5′ end and 3′ end of the constructs or do not have the correct sequences of the barcodes at the 5′ end and the 3′ end of the sequence identifiers. In some embodiments, the Levenshtein distance can be used to cluster clones and thereby correct features. Clones can then be ranked based on confidence in identity assignment.

### Isolation of Target Nucleic Acid Sequences

Aspects of the disclosure are especially useful for isolating nucleic acid sequences of interest from a pool comprising nucleic acid sequences comprising sequences errors. The technology provided herein can embrace any method of non-destructive sequencing. Non-limiting examples of non-destructive sequencing include pyrosequencing, as originally described by Hyman et al., (1988, Anal. Biochem. 74: 324-436) and bead-based sequencing, described for instance by Leamon et al., (2004, Electrophoresis 24: 3769-3777). Non-destructive sequencing also includes methods using cleavable labeled oligonucleotides, as the above described Mitra et al., (2003, Anal. Biochem. 320:55-62) and photocleavable linkers (Seo et al., 2005, PNAS 102: 5926-5933). Methods using reversible terminators are also embraced by the technology provided herein (Metzker et al., 1994, NAR 22: 4259-4267). Further methods for non-destructive sequencing (including single molecule sequencing) are described in U.S. Pat. No. 7,133,782 and U.S. Pat. No. 7,169,560 which are hereby incorporated by reference.

Methods to selectively extract or isolate the correct sequence from the incorrect sequences are provided herein. The term “selective isolation”, as used herein, can involve physical isolation of a desired nucleic acid molecule from others as by selective physical movement of the desired nucleic acid molecule, selective inactivation, destruction, release, or removal of other nucleic acid molecules than the nucleic acid molecule of interest. It should be appreciated that a nucleic acid molecule or library of nucleic acid constructs may include some errors that may result from sequence errors introduced during the oligonucleotides synthesis, the synthesis of the assembly nucleic acids and/or from assembly errors during the assembly reaction. Unwanted nucleic acids may be present in some embodiments. For example, between 0% and 50% (e.g., less than 45%, less than 40%, less than 35%, less than 30%, less than 25%, less than 20%, less than 15%, less than 10%, less than 5% or less than 1%) of the sequences in a library may be unwanted sequences.

In some embodiments, the target having the desired sequence can be recovered using the methods for recovery of the annotated correct target sequences disclosed herein. In some embodiments, the tag sequence pairs for each correct target sequence can be used to amplify by PCR the construct from the sample pool (as illustrated in FIG. 1C, step IV). It should be noted that since the likelihood of the same pair being used for multiple molecules is extremely low, the likelihood to isolate the nucleic acid molecule having the correct sequence is high. Yet in other embodiments, the nucleic acid having the desired sequence can be recovered directly from the sequencer. In some embodiments, the identity of a full length construct can be determined once the pairs of tags are identified. In principle, the location of the full length read (corresponding to a paired end read with the 5′ and 3′ tags) can be determined on the original sequencing flow cell. After locating the cluster on the flow cell surface, molecules can be eluted or otherwise captured from the surface.

In some embodiment, nucleic acids can be sequenced in a sequencing channel. In some embodiments, the nucleic acid constructs can be sequenced in situ on the solid support used in gene synthesis and reused/recycled therefrom. Analysis of the sequence information from the oligonucleotides permits the identification of those nucleic acid molecules that appear to have desirable sequences and those that do not. Such analysis of the sequence information can be qualitative, e.g., providing a positive or negative answer with regard to the presence of one or more sequences of interest (e.g., in stretches of 10 to 120 nucleotides). In some embodiments, target nucleic acid molecules of interest can then be selectively isolated from the rest of the population. The sorting of individual nucleic acid molecules can be facilitated by the use of one or more solid supports (e.g. bead, insoluble polymeric material, planar surface, membrane, porous or non porous surface, chip, or any suitable support, etc. . . . ) to which the nucleic acid molecules can be immobilized. For example, the nucleic acid molecules can be immobilized on a porous surface such as a glass surface or a glass bead. Yet in other examples, the nucleic acid can be immobilized on a flow-through system such as a porous membrane or the like. Nucleic acid molecules determined to have the correct desired sequence can be selectively released or selectively copied.

If the nucleic acid molecules are located in different locations, e.g. in separate wells of a substrate, the nucleic acid molecules can be taken selectively from the wells identified as containing nucleic acid molecules with desirable sequences. For example, in the apparatus of Margulies et al., polony beads are located in individual wells of a fiber-optic slide. Physical extraction of the bead from the appropriate well of the apparatus permits the subsequent amplification or purification of the desirable nucleic acid molecules free of other contaminating nucleic acid molecules. Alternatively, if the nucleic acid molecules are attached to the beads using a selectively cleavable linker, cleavage of the linker (e.g., by increasing the pH in the well to cleave a base-labile linker) followed by extraction of the solvent in the well can be used to selectively isolate the nucleic acid molecules without physical manipulation of the bead. Likewise, if the method of Shendure et al. is used, physical extraction of the beads or of the portions of the gel containing the nucleic acid molecules of interest can be used to selectively isolate desired nucleic acid molecules.

Certain other methods of selective isolation involve the targeting of nucleic acid molecules without a requirement for physical manipulation of a solid support. Such methods can incorporate the use of an optical system to specifically target radiation to individual nucleic acid molecules. In some embodiments, destructive radiation can be selectively targeted against undesired nucleic acid molecules (e.g., using micromirror technology) to destroy or disable them, leaving a population enriched for desired nucleic acid molecules. This enriched population can then be released from solid support and/or amplified, e.g., by PCR.

Example of methods and systems for selectively isolating the desired product (e.g. nucleic acids of interest) can use a laser tweezer or optical tweezer. Laser tweezers have been used for approximately two decades in the fields of biotechnology, medicine and molecular biology to position and manipulate micrometer-sized and submicrometer-sized particles (A. Ashkin, Science, 210, pp. 1081-1088, 1980). By focusing the laser beam on the desired location (e.g. bead, well etc. . . . ) comprising the desired nucleic acid molecule of interest, the desired vessel remain optically trapped while the undesired nucleic acid sequences are eluted. Once all of the undesirable materials are washed off, the optical tweezer can be tuned off allowing the release the desired nucleic acid molecules.

Another method to capture the desirable products is by ablating the undesirable nucleic acids. In some embodiments, a high power laser can be used to generate enough energy to disable, degrade, or destroy the nucleic acid molecules in areas where undesirable materials exist. The area where desirable nucleic acids exist does not receive any destructive energy, hence preserving its contents.

In some embodiments, error-containing nucleic acid constructs can be eliminated. According to some embodiments, the method comprises generating a nucleic acid having oligonucleotide tags at its 5′ end and 3′ end. For example, after assembly of the target sequences (e.g. full length nucleic acid constructs), the target sequences can be barcoded or alternatively, the target sequence can be assembled from a plurality of oligonucleotides designed such that the target sequence has a barcode at its 5′ end and it 3′ end. The tagged target sequence can be fragmented and sequenced using, for example, next-generation sequencing as provided herein. After identification of error-free target sequences, error-free target sequences can be recovered from directly from the next-generation sequencing plate. In some embodiments, error-containing nucleic acids can be eliminated using laser ablation or any suitable method capable of eliminating undesired nucleic acid sequences. The error-free nucleic acid sequences can be eluted from the sequencing plate. Eluted nucleic acid sequences can be amplified using primers that are specific to the target sequences.

In some embodiments, the target polynucleotides can be amplified after obtaining clonal populations. In some embodiments, the target polynucleotide may comprise universal (common to all oligonucleotides), semi-universal (common to at least a portion of the oligonucleotides) or individual or unique primer (specific to each oligonucleotide) binding sites on either the 5′ end or the 3′ end or both. As used herein, the term “universal” primer or primer binding site means that a sequence used to amplify the oligonucleotide is common to all oligonucleotides such that all such oligonucleotides can be amplified using a single set of universal primers. In other circumstances, an oligonucleotide contains a unique primer binding site. As used herein, the term “unique primer binding site” refers to a set of primer recognition sequences that selectively amplifies a subset of oligonucleotides. In yet other circumstances, a target nucleic acid molecule contains both universal and unique amplification sequences, which can optionally be used sequentially.

In some aspects of the disclosure, a binding tag capable of binding error-free nucleic acid molecules or a solid support comprising a binding tag can be added to the error-free nucleic acid sequences. For example, the binding tag, solid support comprising binding tag or solid support capable of binding nucleic acid can be added to locations of the sequencing plate or flow cells identified to include error-free nucleic acid sequences. In some embodiments, the binding tag has a sequence complementary to the target nucleic acid sequence. In some embodiments the binding tag is a double-stranded sequence designed for either hybridization or ligation capture of nucleic acid of interest.

In some embodiments, the solid support can be a bead. In some embodiments, the bead can be disposed onto a substrate. The beads can be disposed on the substrate in a number of ways. Beads, or particles, can be deposited on a surface of a substrate such as a well or flow cell and can be exposed to various reagents and conditions which permit detection of the tag or label. In some embodiments, the binding tags or beads can be deposited by inkjet at specific location of a sequencing plate.

In some embodiments, beads can be derivatized in-situ with binding tags that are complementary to the barcodes or the additional sequences appended to the nucleic acids to capture, and/or enrich, and/or amplify the target nucleic acids identified to have the correct nucleic acid sequences (e.g. error-free nucleic acid). Nucleic acids can be immobilized on the beads by hybridization, covalent attachment, magnetic attachment, affinity attachment and the like. Hybridization is usually performed under stringent conditions. In some embodiments, the binding tags can be universal or generic primers complementary to non-target sequences, for example all barcodes or to appended additional sequences. In some embodiments, each bead can have binding tags capable of binding sequences present both the 5′ end and the 3′ end of the target molecules. Upon binding the target molecules, a loop-like structure is produced. Yet in other embodiments, beads can have a binding tag capable of binding sequences present at the 3′ end of the target molecule. Yet in other embodiments, beads can have a binding tag capable of binding sequences present at the 5′ end of the target molecule.

Beads, such as magnetic or paramagnetic beads, can be added to the each well or arrayed on a solid support. For example, Solid Phase Reversible Immobilization (SPRI) beads from Beckman Coulter can be used. In some embodiments, the pool of constructs can be distributed to the individual wells containing the beads. Additional thermal cycling can be used to enhance capture specificity. Using standard magnetic capture, the solution can then be removed followed by subsequent washing of the conjugated beads Amplification of the desired construct clone can be done either on bead or after release of the captured clone. In some embodiments, the beads can be configured for either hybridization or ligation based capture using double-stranded sequences on the bead.

A variation of the bead-based process can involve a set of flow-sortable encoded beads. Bead-based methods can employ nucleic acid hybridization to a capture probe or attachment on the surface of distinct populations of capture beads. Such encoded beads can be used on a pool of constructs and then sorted into individual wells for downstream amplification, isolation and clean up. While the use of magnetic beads described above can be particularly useful, other methods to separate beads can be envisioned in some aspects of the disclosure. The capture beads may be labeled with a fluorescent moiety which would make the target-capture bead complex fluorescent. For example, the beads can be impregnated with a fluorophore thereby creating distinct populations of beads that can be sorted according to the fluorescence wavelength. The target capture bead complex may be separated by flow cytometry or fluorescence cell sorter. In other embodiments, the beads can vary is size, or in any suitable characteristics allowing the sorting of distinct population of beads. For example, using capture beads having distinct sizes would allow separation by filtering or other particle size separation techniques.

In some embodiments, the flow-sortable encoded beads can be used to isolate the nucleic acid constructs prior to or after post-synthesis release. Such process allows for sorting by construct size, customer etc.

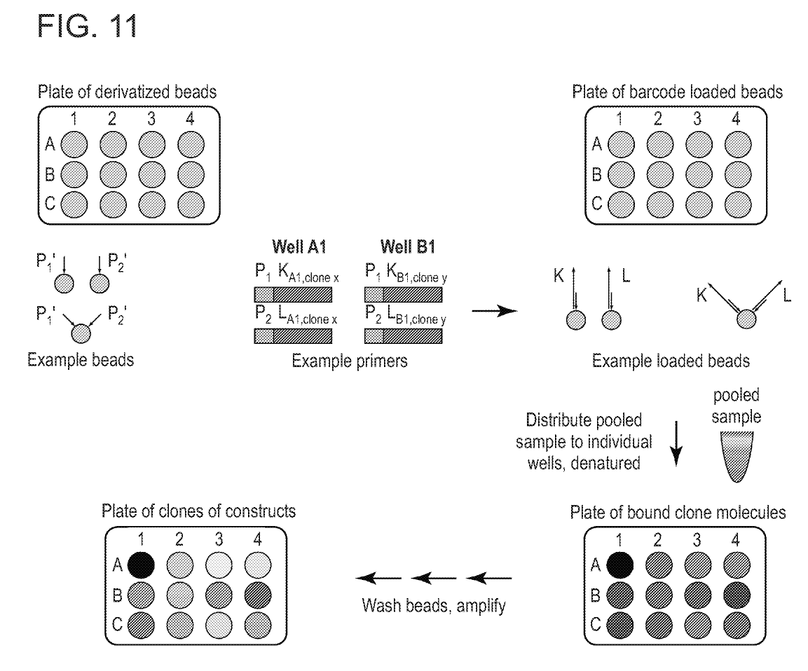

FIG. 11 schematically depicts a non-limiting exemplary bead-based recovery process. In some embodiments, primers can be loaded onto generic beads, for example, magnetic beads. Each bead can be derivatized many times to have many primers bound to it. In some embodiments, derivatization allows to have two or more different primers bound per bead, or to have the same primer bound per bead. Such beads can be distributed in each well of a multi-well plate. Beads can be loaded with barcodes capable of capturing specific nucleic acid molecules, for example by hybridizing a nucleic acid sequence comprising the barcode and a sequence complementary to the primer(s) loaded onto the generic beads. The sample comprising the double-stranded pooled nucleic acids can be subjected to appropriate conditions to render the double-stranded nucleic acids single-stranded. For example, the double-stranded nucleic acids can be subjected to any denaturation conditions known in the art. The pooled single-stranded sample can be distributed across all the wells of a multi-well plate. Under appropriate conditions, the derivatized beads comprising the barcodes can capture specific nucleic acid molecules in each well, based on the exact barcodes (K, L) loaded onto the beads in each well. The beads can then be washed. For example, when using magnetic beads, the beads can be pulled down with a magnet, allowing washing and removal of the solution. In some embodiments, the beads can be washed iteratively. The nucleic acids that remained bound on the beads can then amplified using PCR to produce individual clones in each well of the multi-well construct plate.

In other aspects of the disclosure, nanopore sequencing can be used to sequence individual nucleic acid strand at single nucleotide level. One of skill in the art would appreciate that nanopore sequencing has the advantage of minimal sample preparation, sequence readout that does not require nucleotides, polymerases or ligases, and the potential of very long read-lengths. However, nanopore sequencing can have relatively high error rates (˜10% error per base). In some embodiments, the nanopore sequencing device comprises a shuntable microfluidic flow valve to recycle the full length nucleic acid construct so as to allow for multiple sequencing passes. In some embodiments, the nanopores can be connected in series with a shuntable microfluidic flow valve such that full length nucleic acid construct can be shunted back to the nanopore several times to allow for multiple sequencing passes. Using these configurations, the full length nucleic acid molecules can be sequenced two or more times. Resulting error-free nucleic acid sequences may be shunted to a collection well for recovery and use.

In some aspects of the disclosure, alternative preparative sequencing methods are provided herein. The methods comprise circularizing the target nucleic acid (e.g. the full length target nucleic acid) using double-ended primers capable of binding the 5′ end and the 3′ end of the target nucleic acids. In some embodiments, the double-ended primers have sequences complementary to the 5′ end and the 3′ end barcodes. Nucleases can be added so as to degrade the linear nucleic acid, thus locking-in the desired constructs. Optionally, the target nucleic acid can be amplified using primers specific to the target nucleic acids.

### Inverted In Vitro Cloning

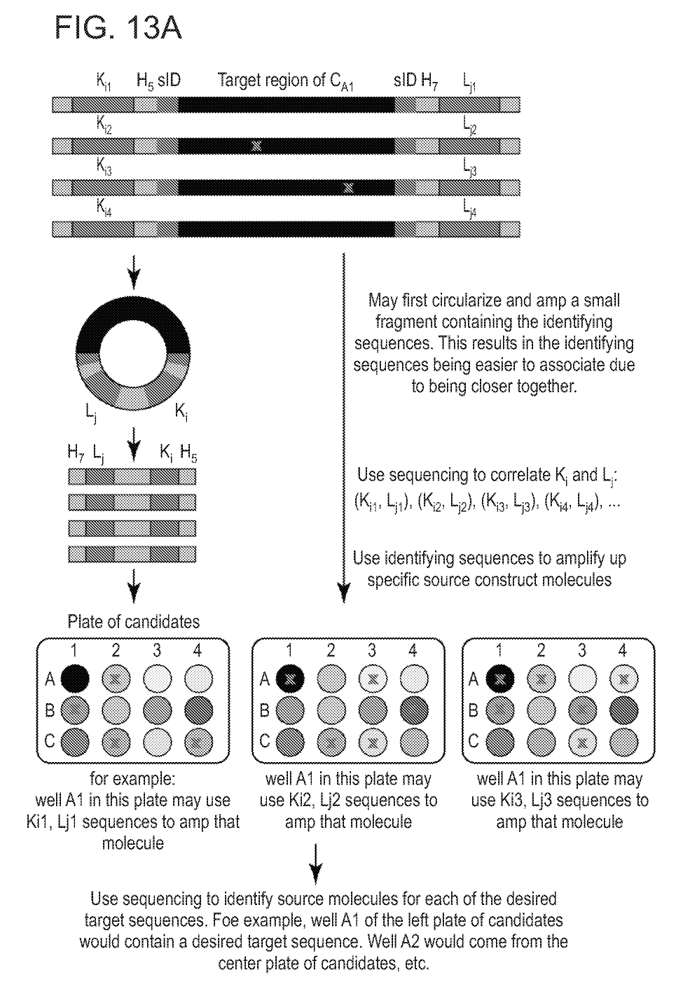

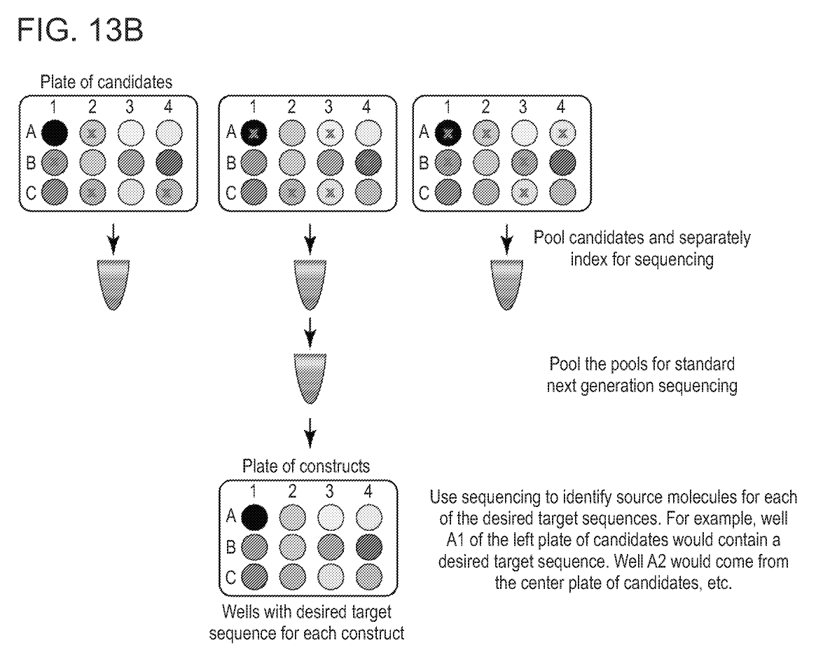

In some aspects of the disclosure, methods are provided to isolate and/or recover a sequence-verified nucleic acid of interest. The methods described herein may be used to recover for example, error-free nucleic acid sequences of interest from a nucleic acid library or a pool of nucleic acid sequences. The nucleic acid library or the pool of nucleic acid sequences may include one or more target nucleic acid sequences of interest (e.g. N genes). In some embodiments, the library of nucleic acid sequences can include constructs assembled from oligonucleotides or nucleic acid fragments. A plurality of barcoded constructs can be assembled as described herein. In some embodiments, the plurality of constructs can be assembled and barcoded using a library of barcodes such that each nucleic acid construct can be tagged with a unique barcode at each end. Yet in other embodiments, the plurality of constructs can be assembled from a plurality of internal target sequence fragments and unique barcode sequences. For example, the library of nucleic acid sequences can comprise M copies of N different target nucleic acid sequences. For instance 100 copies of 96 target sequences, and the library of barcodes can have 316 different barcodes for a combinatorics of 100,000. In some embodiments, the library of barcodes can have common amplification sequences (e.g. common primer binding sequences) on the outside of the barcodes. In some embodiments, if necessary, the pool of barcoded constructs can be amplified using the common amplification tags such as to have an appropriate concentration of nucleic acids for next generation sequencing. In some embodiments, the barcoded constructs can be subjected to sequencing reactions from both ends to obtain short paired end reads. In some embodiments, and as illustrated in FIG. 13A, the barcoded constructs of the pool of constructs can be circularized so as to get a barcode association which is independent of the length of the nucleic acid constructs. This way, a small nucleic acid fragment containing the identifying sequences such as barcodes Kᵢ and Lⱼ or oligonucleotide tags can be amplified. Identifying sequences are subjected to sequencing to correlate Kᵢ and Lⱼ (Kᵢ₁, Lⱼ₁), (Kᵢ₂, Lⱼ₂) etc. . . . For example, sequencing of the identifying sequences can result in C clones having the target sequence according to the identity of their corresponding unique pair of identifying sequences. The identifying sequences can then be used to amplify the C specific source construct molecules in separate wells of a microtiter plate as illustrated in FIG. 13A. For example, if C=8 clones, 8 plates of N target nucleic acid sequences (e.g. 96 genes) can be provided, each plate having a different index tag (FIG. 13B). Source molecules (C*N) can be digested using Nextera™ tagmentation and sequenced using MiSeq®, HiSeq® or higher throughput next generation sequencing platforms to identify the correct target sequences. Sequencing data can be used to identify the target nucleic acid sequence, and sort the sequence-verified nucleic acid of interest. For example, as illustrated in FIG. 13A, well A1 of the left plate of candidates would contain the sequence-verified nucleic acid of interest. The identified clone can then be recovered from the well identified to have the sequence-verified nucleic acid of interest.

### Determination of Barcode Pair Information

In some embodiments, and as described herein, the barcode pairs can be defined by sequencing full length molecules. Sequencing from both ends gives the required pairing information. For the most effective determination of barcode pairs using full length sequencing method, multiple Nextera™ tagmentation reactions, where the amount of Nextera™ enzyme is varied. These individual reactions can be processed in parallel and sequenced using MiSeq® at the same time using separate indexes. The read information can then be combined and processed as a whole. Using such process design allows for the identification of error-free molecules that can be subsequently captured by amplification. However due to the length limitation of the MiSeq® sequencing (e.g. poor sequencing of nucleic acids longer than ˜1000 bps), barcode pairing using this method can be inefficient for constructs greater than 1000 bps.

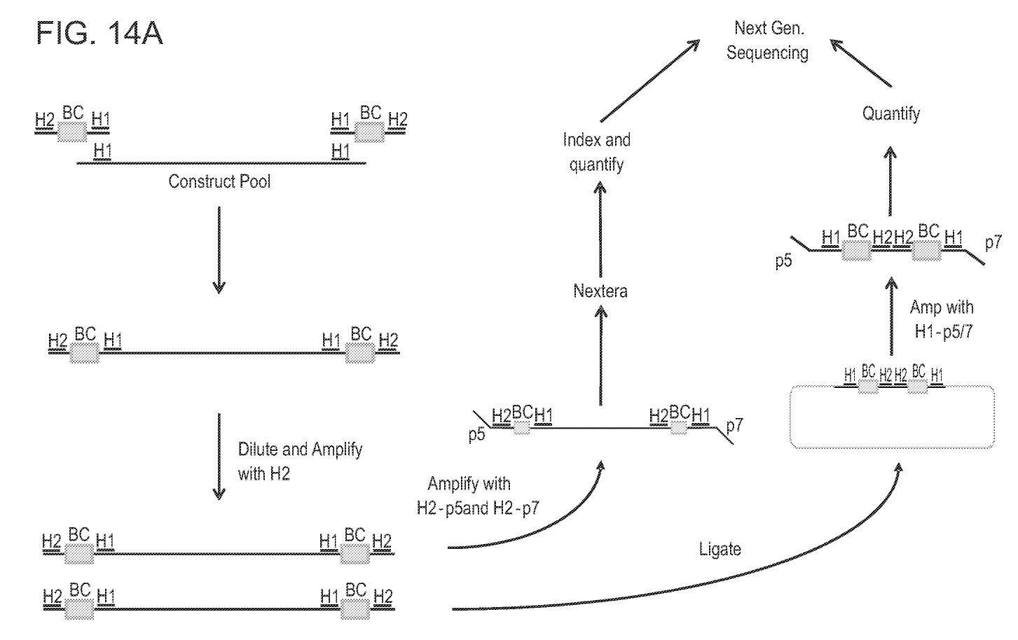

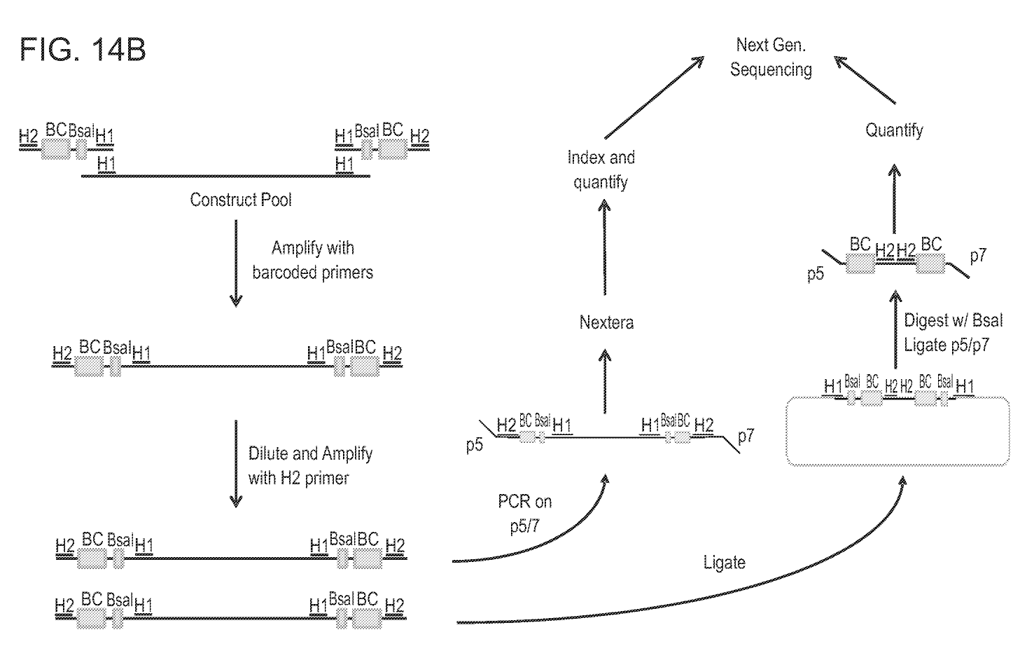

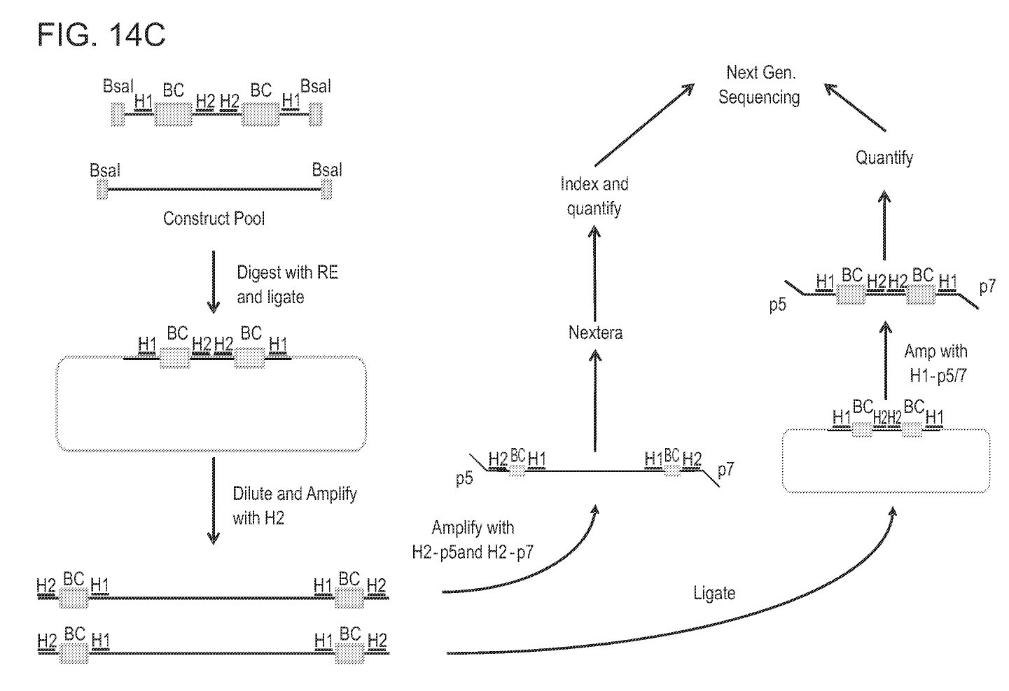

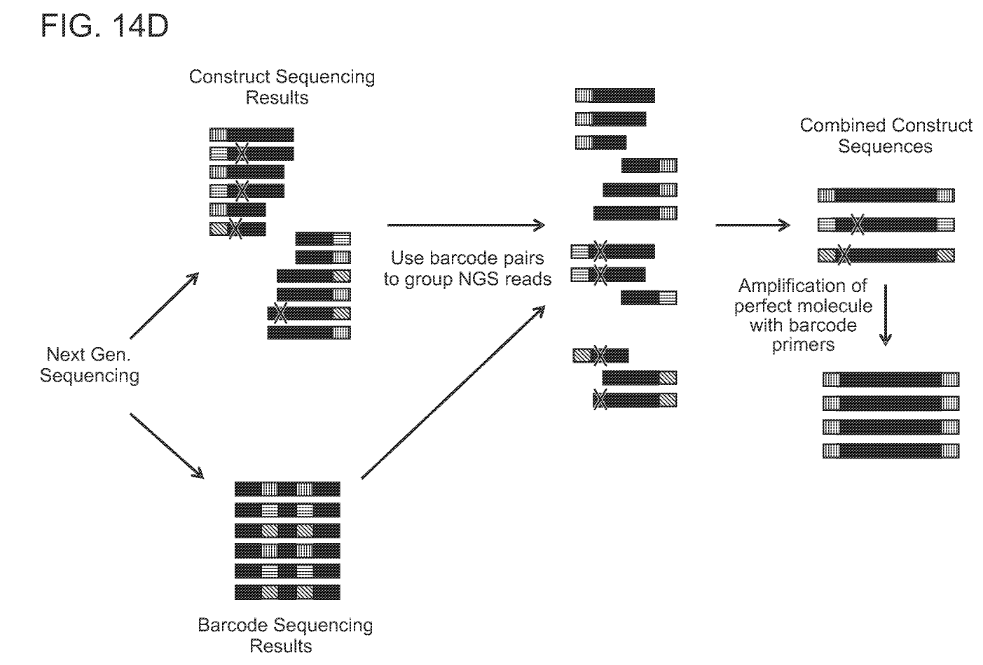

The barcode pair information, according to some embodiments, can be determined according to the methods described in FIGS. 14A-14D. FIGS. 14A and 14B illustrate different methods allowing the the barcoded ends of the molecules to be brought together by blunt end ligation of the constructs into circles. In both concepts, barcodes can be added to the constructs via PCR, using sequence H1 as priming sites. After dilution and amplification with H2 primers, the construct pools can be split into two parallel paths. One part can be amplified with H2 primers with the p5 and p7 sequences necessary for sequencing on the MiSeq®. The amplified constructs can be fragmented by Nextera™ based cleavage and subsequently sequenced using MiSeq®. The second path is focused on determining the barcode pairing information. Referring to FIG. 14A, the barcode pairs can be amplified and sequenced. Referring to FIG. 14B, the barcode pairs can be cut out of the circle by restriction digest and subsequently sequenced. Using the methods described herein, the end barcode pairs can be associated in a manner that is independent of the length of the construct being sequenced.

FIG. 14C illustrates a different method of attaching barcodes to the synthesized constructs. According to some embodiments, restriction enzymes, such as BsaI or any suitable restriction enzyme, can be used to open compatible nucleic acid overhangs which can then be used to ligate paired barcode molecules to the constructs, resulting in circular constructs. The pool of circular constructs can then diluted and amplified with primer H2. The constructs can then be processed as shown in either FIG. 14A or FIG. 14B. FIG. 14D shows a non-limiting embodiment using parallel sequencing of constructs and the isolated barcode pairs to identify the correct molecule for subsequent capture by amplification.

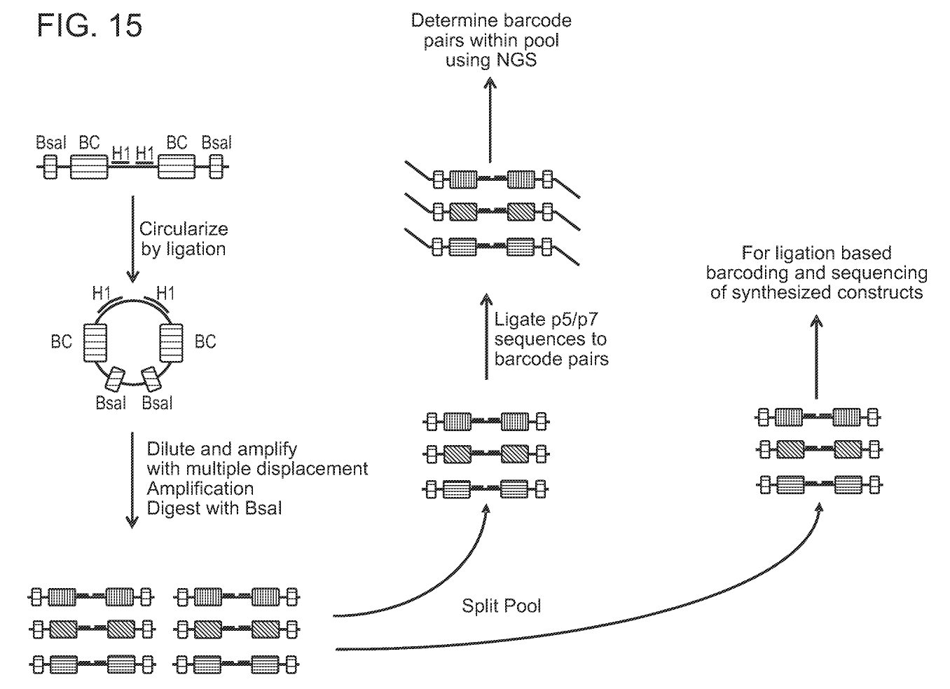

According to some embodiments, the barcode pairs can be generated as a pool of molecules, each with a single pair of barcodes. Referring to FIG. 15, these molecules can be circularized and diluted to an appropriate level, which can be defined by the appropriate total number of barcodes. For example, the number of barcodes can be 10̂5 or 10̂6. The diluted barcodes can then be amplified using multiple displacement amplification to generate multiple copies of each barcode. The resulting pool of barcodes can then split into two. A first portion can be used in barcoding synthesized constructs. The second portion can be sequenced using next generation sequencing. The sequencing data will give the barcode-barcode associations within the pool. With appropriate sequencing, the pool can be defined to completion. It should be appreciate that when sequencing the constructs using such pool, the barcode associations are already known, removing the need for processes outlined in FIGS. 14A-14D.

### Applications

Aspects of the disclosure may be useful for a range of applications involving the production and/or use of synthetic nucleic acids. As described herein, the disclosure provides methods for producing synthetic nucleic acids having the desired sequence with increased efficiency. The resulting nucleic acids may be amplified in vitro (e.g., using PCR, LCR, or any suitable amplification technique), amplified in vivo (e.g., via cloning into a suitable vector), isolated and/or purified. An assembled nucleic acid (alone or cloned into a vector) may be transformed into a host cell (e.g., a prokaryotic, eukaryotic, insect, mammalian, or other host cell). In some embodiments, the host cell may be used to propagate the nucleic acid. In certain embodiments, the nucleic acid may be integrated into the genome of the host cell. In some embodiments, the nucleic acid may replace a corresponding nucleic acid region on the genome of the cell (e.g., via homologous recombination). Accordingly, nucleic acids may be used to produce recombinant organisms. In some embodiments, a target nucleic acid may be an entire genome or large fragments of a genome that are used to replace all or part of the genome of a host organism. Recombinant organisms also may be used for a variety of research, industrial, agricultural, and/or medical applications.

Many of the techniques described herein can be used together, applying suitable assembly techniques at one or more points to produce long nucleic acid molecules. For example, ligase-based assembly may be used to assemble oligonucleotide duplexes and nucleic acid fragments of less than 100 to more than 10,000 base pairs in length (e.g., 100 mers to 500 mers, 500 mers to 1,000 mers, 1,000 mers to 5,000 mers, 5,000 mers to 10,000 mers, 25,000 mers, 50,000 mers, 75,000 mers, 100,000 mers, etc.). In an exemplary embodiment, methods described herein may be used during the assembly of an entire genome (or a large fragment thereof, e.g., about 10%, 20%, 30%, 40%, 50%, 60%, 70%, 80%, 90%, or more) of an organism (e.g., of a viral, bacterial, yeast, or other prokaryotic or eukaryotic organism), optionally incorporating specific modifications into the sequence at one or more desired locations.

Any of the nucleic acid products (e.g., including nucleic acids that are amplified, cloned, purified, isolated, etc.) may be packaged in any suitable format (e.g., in a stable buffer, lyophilized, etc.) for storage and/or shipping (e.g., for shipping to a distribution center or to a customer). Similarly, any of the host cells (e.g., cells transformed with a vector or having a modified genome) may be prepared in a suitable buffer for storage and or transport (e.g., for distribution to a customer). In some embodiments, cells may be frozen. However, other stable cell preparations also may be used.

Host cells may be grown and expanded in culture. Host cells may be used for expressing one or more RNAs or polypeptides of interest (e.g., therapeutic, industrial, agricultural, and/or medical proteins). The expressed polypeptides may be natural polypeptides or non-natural polypeptides. The polypeptides may be isolated or purified for subsequent use.

Accordingly, nucleic acid molecules generated using methods of the disclosure can be incorporated into a vector. The vector may be a cloning vector or an expression vector. In some embodiments, the vector may be a viral vector. A viral vector may comprise nucleic acid sequences capable of infecting target cells. Similarly, in some embodiments, a prokaryotic expression vector operably linked to an appropriate promoter system can be used to transform target cells. In other embodiments, a eukaryotic vector operably linked to an appropriate promoter system can be used to transfect target cells or tissues.

Transcription and/or translation of the constructs described herein may be carried out in vitro (i.e. using cell-free systems) or in vivo (i.e. expressed in cells). In some embodiments, cell lysates may be prepared. In certain embodiments, expressed RNAs or polypeptides may be isolated or purified. Nucleic acids of the disclosure also may be used to add detection and/or purification tags to expressed polypeptides or fragments thereof. Examples of polypeptide-based fusion/tag include, but are not limited to, hexa-histidine (His⁶) Myc and HA, and other polypeptides with utility, such as GFP₅ GST, MBP, chitin and the like. In some embodiments, polypeptides may comprise one or more unnatural amino acid residue(s).

In some embodiments, antibodies can be made against polypeptides or fragment(s) thereof encoded by one or more synthetic nucleic acids. In certain embodiments, synthetic nucleic acids may be provided as libraries for screening in research and development (e.g., to identify potential therapeutic proteins or peptides, to identify potential protein targets for drug development, etc.) In some embodiments, a synthetic nucleic acid may be used as a therapeutic (e.g., for gene therapy, or for gene regulation). For example, a synthetic nucleic acid may be administered to a patient in an amount sufficient to express a therapeutic amount of a protein. In other embodiments, a synthetic nucleic acid may be administered to a patient in an amount sufficient to regulate (e.g., down-regulate) the expression of a gene.

It should be appreciated that different acts or embodiments described herein may be performed independently and may be performed at different locations in the United States or outside the United States. For example, each of the acts of receiving an order for a target nucleic acid, analyzing a target nucleic acid sequence, designing one or more starting nucleic acids (e.g., oligonucleotides), synthesizing starting nucleic acid(s), purifying starting nucleic acid(s), assembling starting nucleic acid(s), isolating assembled nucleic acid(s), confirming the sequence of assembled nucleic acid(s), manipulating assembled nucleic acid(s) (e.g., amplifying, cloning, inserting into a host genome, etc.), and any other acts or any parts of these acts may be performed independently either at one location or at different sites within the United States or outside the United States. In some embodiments, an assembly procedure may involve a combination of acts that are performed at one site (in the United States or outside the United States) and acts that are performed at one or more remote sites (within the United States or outside the United States).

### Automated Applications

Aspects of the methods and devices provided herein may include automating one or more acts described herein. In some embodiments, one or more steps of an amplification and/or assembly reaction may be automated using one or more automated sample handling devices (e.g., one or more automated liquid or fluid handling devices). Automated devices and procedures may be used to deliver reaction reagents, including one or more of the following: starting nucleic acids, buffers, enzymes (e.g., one or more ligases and/or polymerases), nucleotides, salts, and any other suitable agents such as stabilizing agents. Automated devices and procedures also may be used to control the reaction conditions. For example, an automated thermal cycler may be used to control reaction temperatures and any temperature cycles that may be used. In some embodiments, a scanning laser may be automated to provide one or more reaction temperatures or temperature cycles suitable for incubating polynucleotides. Similarly, subsequent analysis of assembled polynucleotide products may be automated. For example, sequencing may be automated using a sequencing device and automated sequencing protocols. Additional steps (e.g., amplification, cloning, etc.) also may be automated using one or more appropriate devices and related protocols. It should be appreciated that one or more of the device or device components described herein may be combined in a system (e.g., a robotic system) or in a micro-environment (e.g., a micro-fluidic reaction chamber). Assembly reaction mixtures (e.g., liquid reaction samples) may be transferred from one component of the system to another using automated devices and procedures (e.g., robotic manipulation and/or transfer of samples and/or sample containers, including automated pipetting devices, micro-systems, etc.). The system and any components thereof may be controlled by a control system.

Accordingly, method steps and/or aspects of the devices provided herein may be automated using, for example, a computer system (e.g., a computer controlled system). A computer system on which aspects of the technology provided herein can be implemented may include a computer for any type of processing (e.g., sequence analysis and/or automated device control as described herein). However, it should be appreciated that certain processing steps may be provided by one or more of the automated devices that are part of the assembly system. In some embodiments, a computer system may include two or more computers. For example, one computer may be coupled, via a network, to a second computer. One computer may perform sequence analysis. The second computer may control one or more of the automated synthesis and assembly devices in the system. In other aspects, additional computers may be included in the network to control one or more of the analysis or processing acts. Each computer may include a memory and processor. The computers can take any form, as the aspects of the technology provided herein are not limited to being implemented on any particular computer platform. Similarly, the network can take any form, including a private network or a public network (e.g., the Internet). Display devices can be associated with one or more of the devices and computers. Alternatively, or in addition, a display device may be located at a remote site and connected for displaying the output of an analysis in accordance with the technology provided herein. Connections between the different components of the system may be via wire, optical fiber, wireless transmission, satellite transmission, any other suitable transmission, or any combination of two or more of the above.

Each of the different aspects, embodiments, or acts of the technology provided herein can be independently automated and implemented in any of numerous ways. For example, each aspect, embodiment, or act can be independently implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the technology provided herein comprises at least one computer-readable medium (e.g., a computer memory, a floppy disk, a compact disk, a tape, etc.) encoded with a computer program (i.e., a plurality of instructions), which, when executed on a processor, performs one or more of the above-discussed functions of the technology provided herein. The computer-readable medium can be transportable such that the program stored thereon can be loaded onto any computer system resource to implement one or more functions of the technology provided herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the technology provided herein.

It should be appreciated that in accordance with several embodiments of the technology provided herein wherein processes are stored in a computer readable medium, the computer implemented processes may, during the course of their execution, receive input manually (e.g., from a user).

Accordingly, overall system-level control of the assembly devices or components described herein may be performed by a system controller which may provide control signals to the associated nucleic acid synthesizers, liquid handling devices, thermal cyclers, sequencing devices, associated robotic components, as well as other suitable systems for performing the desired input/output or other control functions. Thus, the system controller along with any device controllers together form a controller that controls the operation of a nucleic acid assembly system. The controller may include a general purpose data processing system, which can be a general purpose computer, or network of general purpose computers, and other associated devices, including communications devices, modems, and/or other circuitry or components to perform the desired input/output or other functions. The controller can also be implemented, at least in part, as a single special purpose integrated circuit (e.g., ASIC) or an array of ASICs, each having a main or central processor section for overall, system-level control, and separate sections dedicated to performing various different specific computations, functions and other processes under the control of the central processor section. The controller can also be implemented using a plurality of separate dedicated programmable integrated or other electronic circuits or devices, e.g., hard wired electronic or logic circuits such as discrete element circuits or programmable logic devices. The controller can also include any other components or devices, such as user input/output devices (monitors, displays, printers, a keyboard, a user pointing device, touch screen, or other user interface, etc.), data storage devices, drive motors, linkages, valve controllers, robotic devices, vacuum and other pumps, pressure sensors, detectors, power supplies, pulse sources, communication devices or other electronic circuitry or components, and so on. The controller also may control operation of other portions of a system, such as automated client order processing, quality control, packaging, shipping, billing, etc., to perform other suitable functions known in the art but not described in detail herein.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The following examples are set forth as being representative of the present disclosure. These examples are not to be construed as limiting the scope of the disclosure as these and other equivalent embodiments will be apparent in view of the present disclosure, figures and accompanying claims.

### EXAMPLES

### Example 1

The methods described herein and illustrated in FIG. 1A-C allow for the identification of target nucleic acids having the correct desired sequence from a plate of having a plurality of distinct nucleic acid constructs, each plurality of nucleic acid constructs comprising a mixture of correct and incorrect sequences.

In step I, FIG. 1A, a plurality of constructs (C_{A1}-C_{An}, . . . C_{N1}-C_{Nn}) is provided within separate wells of a microplate, each well comprising a mixture of correct and incorrect sequence sites. Each construct can have a target region flanked at the 5′ end with a construct specific region X and a common region or adaptor A and at the 3′ end a construct specific region Y and a common region or adaptor B.

In step II, FIG. 1A, each of the construct mixture can be diluted to a limited number of molecules (about 100-1000) such as each well of the plate comprise normalized mixture of molecules. Each of the dilutions can be mixed and pooled together into one tube.

In step III, FIG. 1A, the plurality of molecules is tagged with pairs of primers (P1, P2) and a large library of nucleotide tags or barcodes (K,L) by ligation or polymerase chain reaction. The methods described herein allow for each molecule to be tagged with a unique pair of barcodes (K, L) to distinguish the molecule from the other molecules in the pool. For example, each well can comprise about 100 molecules and each molecule can be tagged with a unique K-L tag (e.g. K₁-L₁; Kⱼ-Lⱼ, . . . K₁₀₀-L₁₀₀). The entire sample can be amplified to generate enough material for sequencing and the preparative recovery.

In step IV, FIG. 1B, the sample is then split, with the bulk of the sample undergoing Nextera™ tagmentation. The tagmentation reaction can be optimized to make under two breakages per molecule, ensuring that the bulk of the molecules contain one of the tag barcodes and a partial length of the construct target region. The reserved portion of the sample that did not undergo tagmentation, is mixed back in and prepped for sequencing. Two example molecules with one break are shown, each splitting two to sequencing fragments with a tag from the 5′ or 3′ end. For example, as illustrated in FIG. 1B, molecule b can be splitted in two to generate b1 and b2.

In step V, FIG. 1B, the full length molecules generate paired reads which map the tag pairs (Kj, Lj) to individual clonal construct molecules (for example construct C₁, clone j in well 1). The Nextera™ tagmented paired reads generate one sequence with a tag for identification, and another sequence internal to the construct target region. With high throughput sequencing, enough coverage can be generated to reconstruct the consensus sequence of each tag pair construct and determine if the sequence is correct. For example, as illustrated in FIG. 1B, each fragment in sequencing generates two reads (a paired read). Molecule “a” generates reads with associate a unique barcode K_{A1-x} with a unique barcode L_{A1-x} No other molecule should have the same combination. If two molecules from the same construct have a common barcode, the data is discarded due to the ambiguity of the source molecule for those reads. Fragments b1, b2, c1, c2 etc. are identified by one read of the paired read with the barcode. The other read is used to make consensus sequence of internal regions of the molecule. The consensus sequence fro each clone is compared with the desired sequence. The example shows results from well A1 in which clone x is correct, but clone y and z are incorrect. Similar results for each of the original constructs pooled together can be obtained in parallel from the sequencing results.

In step VI, FIG. 1C, the correct construct sequences is amplified using a pair of primers in each well which have the unique tag sequences from the tag pair corresponding to the correct nucleic acid clone. Each clone can be amplified with the tagged pool as a template in individual wells. This allows for the generation of a plate of cloned constructs, each well containing a different desired sequence with each molecule having the correct sequence. As illustrated in FIG. 1C, the molecules in each well are in vitro clones of the original constructs, with flanking sequences corresponding to the barcode combination (K,L) used to amplify the clones having the correct predetermined sequence.

### Example 2

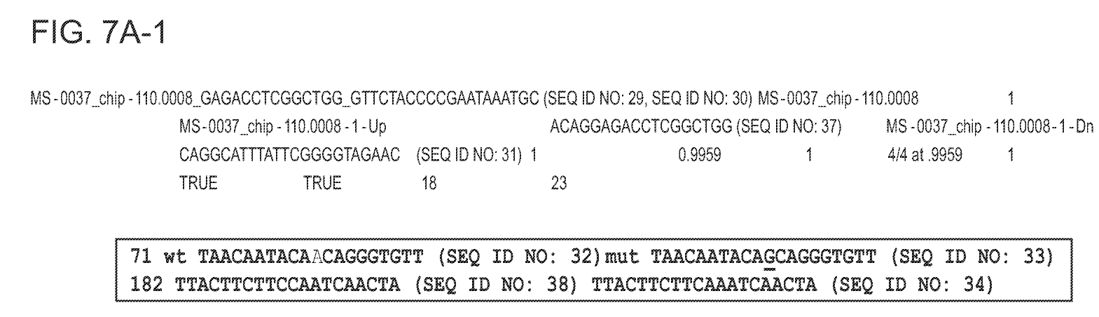

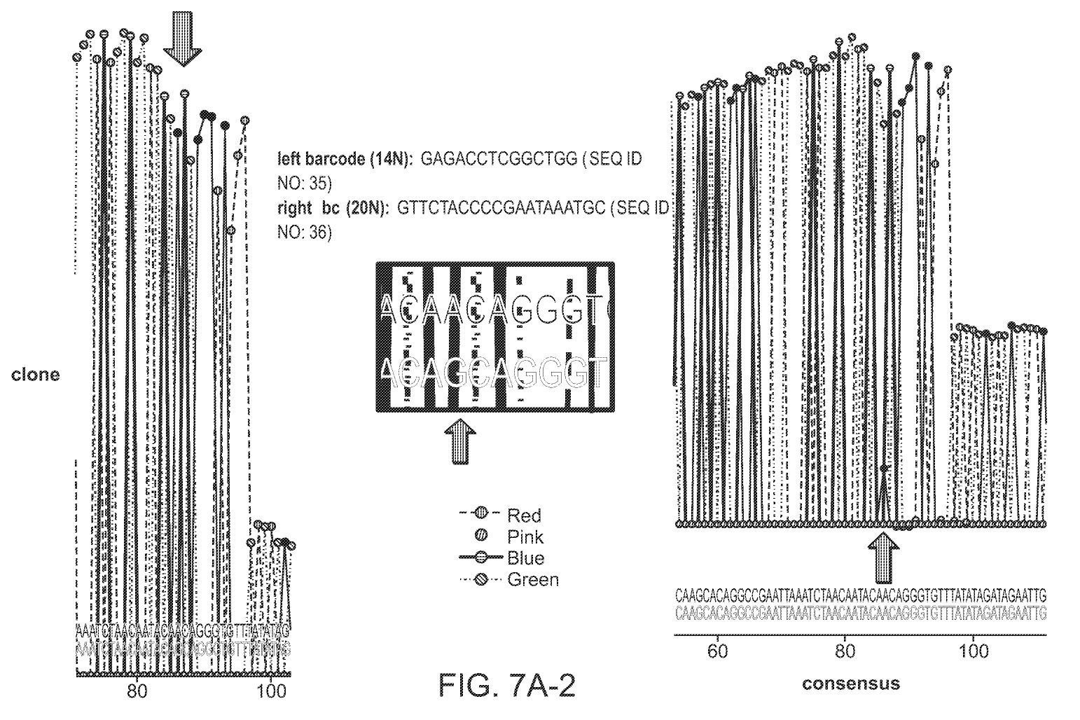

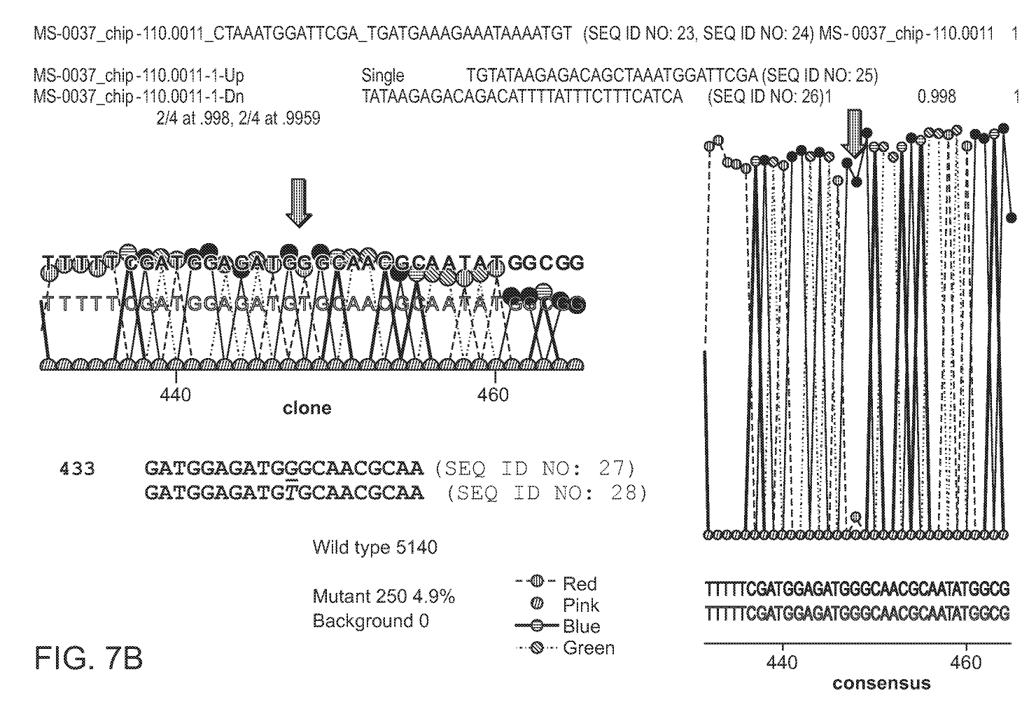

The foregoing methods of in vitro cloning can be extremely effective at distinguishing individual source molecules. A consensus sequence (from all the source molecules of one construct) can have small competing signals from individual source molecules with errors at a position. In some embodiments, the consensus sequence can be compared with the trace from that individual source molecule with the error. In most of the cases, the source molecule can be cleanly called as an error, with no competing signal from the (large) background of the correct base. FIGS. 7A and 7B illustrate an example of effective source molecule separation. On the right side is a consensus trace of all reads of a particular construct at a certain location. As illustrated in FIGS. 7A-B, where there is a “mutation” or “error” signal, quite small relative to the whole population, that mutation/error stems from a single clone (source molecule). On the left side is a consensus trace of all reads of the same construct but from a particular barcode pair (i.e. clone). The same position is shown, which contains only the “mutation” signal and no signal from the wild-type/reference background. Thus the two signals are completely separable and correspond to individual source molecules which are distinguished.

### Example 3

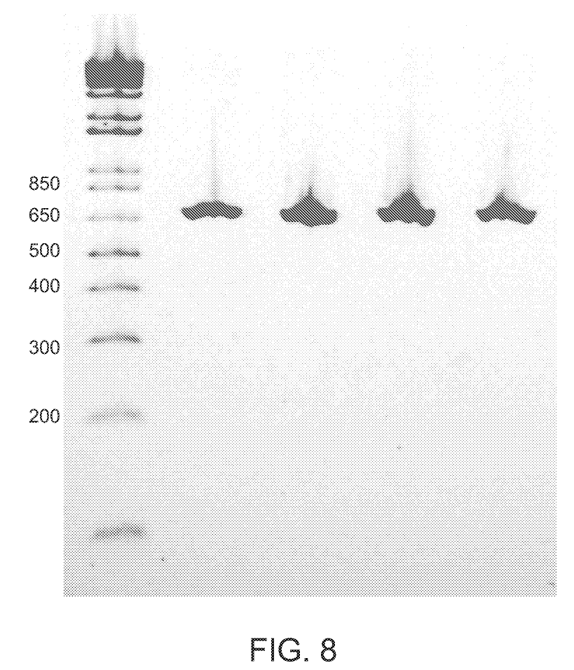

FIG. 8 illustrates the use of coded barcodes to isolate or fish out nucleic acids having the predetermined sequences. In an exemplary embodiment, the 5′ barcode is 14N and the 3′ barcode is 20N. Primers (also referred herein as fish-out primers) were used for isolation of targets (chip-110.0001) as illustrated in FIG. 8. Each barcode pair (left barcode is in bold as illustrated below) was used to make primers. Clone A uses primer sequences 1 & 2; clone B uses 3 & 4, etc . . . . The target molecule was recovered very cleanly using PCR with the fish-out primers.

SEQ ID NO: 1, SEQ ID NO: 2)
chip-110.0001_ACTCACCTCGTTTC_CCTTATAAGCATGTCTCATA
Primer 1
(SEQ ID NO: 3)
AGAGACAGACTCACCTCGTTTC
Primer 2
(SEQ ID NO: 4)
GAGACAGTATGAGACATGCTTATAAGG
(SEQ ID No. 5, SEQ ID NO: 6)
chip-110.0001_GCCGCCGCTGGGGC_CCTCCCCACGCTCTCTAGCC
Primer 3
(SEQ ID NO: 7)
GGCCGCCGCTGGGGC
Primer 4
(SEQ ID NO: 8)
ACAGGGCTAGAGAGCGTGGGGAGG
(SEQ ID NO: 9, SEQ ID NO: 10)
chip-110.0001_GGAGCGATCACCAT_TAGACGTTCATGGTACATAC
Primer 5
(SEQ ID NO: 11)
ACAGGGAGCGATCACCAT
Primer 6
(SEQ ID NO: 12)
ACAGGTATGTACCATGAACGTCTA
(SEQ ID NO: 13, SEQ ID NO: 14)
chip-110.0001_CGGAGTGCTGGGAT_CCTTTGTGGTCATGAGTTTG
Primer 7
(SEQ ID NO: 15)
AGCGGAGTGCTGGGAT
Primer 8
(SEQ ID NO: 16)
AGCAAACTCATGACCACAAAGG

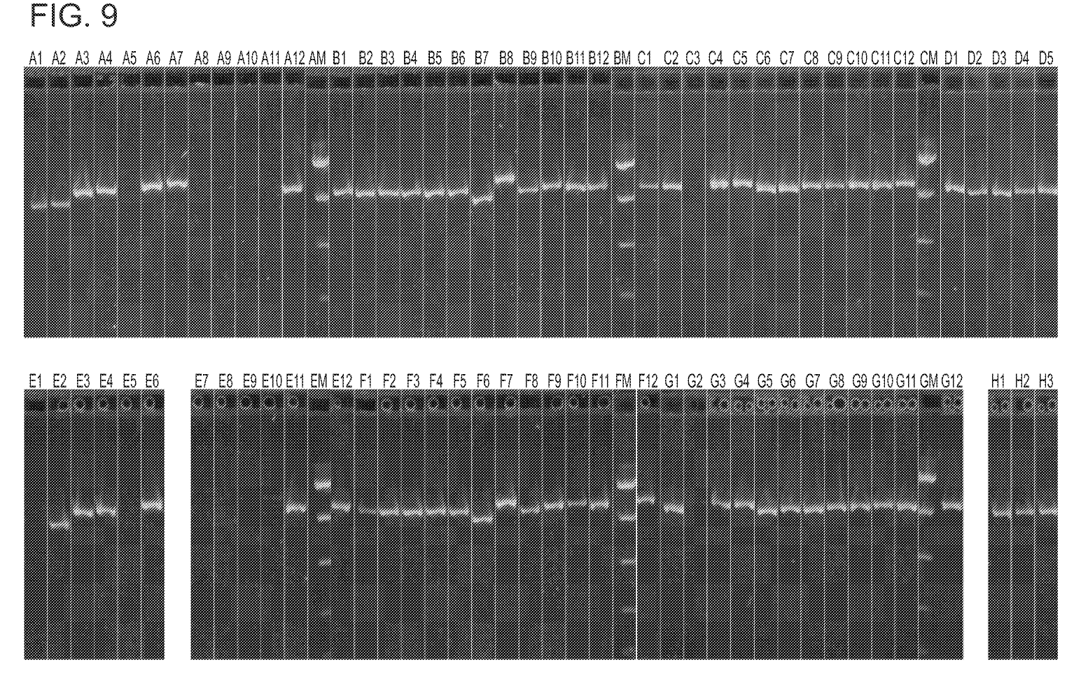

As illustrated in FIG. 9, 54 constructs ranging in size from about 650 to about 1100 bps were normalized and pooled together. The barcodes were attached by polymerase chain reaction using the handle sequences on each construct (5′: CATCAACGTTCATGTCGCGC (SEQ ID NO: 17), 3′: CCTTGGGTGCTCGCAGTAAA (SEQ ID NO: 18)). The barcoded primers were composed of a common region for Illumina sequencing preparation, a degenerate portion for the barcode, and the handle sequences shown above. The degenerate portion of the 5′ barcode was designed to have 14N and the degenerate portion of the 3′ barcode was designed to have a 20N. The 5′ barcodes primer was composed of the following sequences: TCGTCGGCAGCGTC (SEQ ID NO: 19), AGATGTGTATAAGAGACAG (SEQ ID NO: 20), and CATCAACGTTCATGTCGCGC (SEQ ID NO: 21). The 3′ barcoded primer was composed of the following sequences: GTCTCGTGGGCTCGG (SEQ ID NO: 22), AGATGTGTATAAGAGACAG (SEQ ID NO: 23), and CCTTGGGTGCTCGCAGTAAA (SEQ ID NO: 24).

Polymerase chain reaction (PCR) was carried out using KOD polymerase for 5 cycles. The resulting mixture was purified using SPRI beads to remove short products and primers. The pooled sample was then diluted to a factor of 512,000 fold using 8 fold dilutions of a 1000× fold initial dilution. The pooled sample was used as a template in a PCR reaction, using KOD polymerase and using primers corresponding to the 5′ common region of the primers for the previous PCR. After 30 cycles, the sample was again purified using SPRI beads to remove short products, primers, and protein. The sample at this stage is called the “fish-out template”.

The Nextera™ tagmentation reaction was performed as prescribed in the Illumina manual, but with increased input DNA amount (150 ng). The tagmentation reaction was cleaned with a Zymo purification kit (as recommended in the Illumina manual). The sample was then indexed, also according to the Illumina manual, and SPRI cleaned again.

The resulting DNA library was quantified by qPCR using the KAPA Sybr® Library quantification kit (Kapa Biosystems), as described in its manual. The resulting standard curve and titration curves were used to convert DNA concentrations into nM scale. A 2 nM or 4 nM concentration aliquot of the sample was prepared for MiSeq® sequencing as described in the Illumina manual and loaded on the instrument at about 15 pM.

FIG. 9 illustrates the demonstration for half a plate: 851 called clones, spanning 41 constructs (includes both perfects and called mutations). 80 pairs of primers (about 2 per construct) were generated. 67 of 80 (84%) of clone isolations were successful. Four clones were sent of each for Sanger sequencing. The barcodes used for this demonstration were the coded barcodes as described above.

### Informatics Analysis:

The sequencing reads were taken from the MiSeq® instrument and aligned to reference sequences using Smith-Waterman alignment for the handle sequences. Barcodes from aligned reads were read by taking the sequence adjacent to the handle sequence, thus building a correlation of barcodes to reads. Read pairs were determined where the first read contained the 5′ barcode and the second read contained the 3′ barcode. These associations were thresholded and scored, to make pairs of high confidence. Those were then used to form subset read populations containing all reads which contained either barcode, and then aligned to the reference sequence to call a consensus sequence for that clone. Traces were generated showing the number of reads called for each position (and their base identity).

Barcode pairs which generated a perfect consensus sequence to the reference were then used to make primers, containing as much of the barcode sequence as possible, having suitable melting temperatures and desired other features. The primers were used in a PCR reaction using KOD polymerase with the template being a small dilution amount of the “fish-out template”.

### Example 4

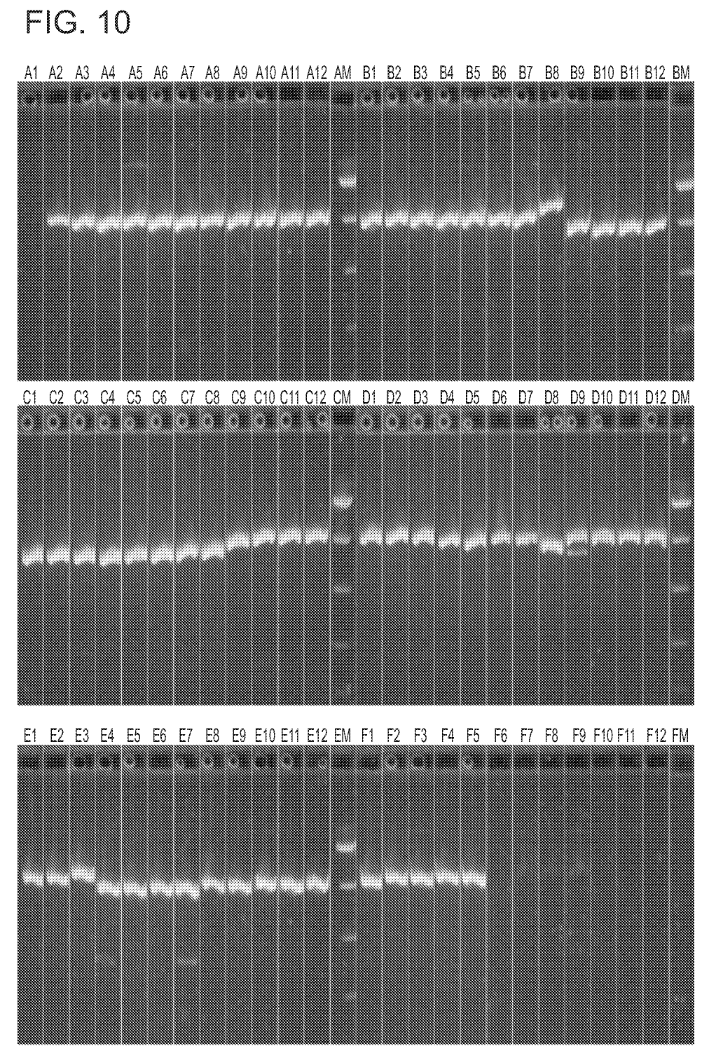

In this full plate example, 87 constructs ranging in size from ˜700 to ˜1200 bp were pooled together. There were 2052 called clones spanning 71 constructs (82%) with 1387 called perfect (68%). Perfects called spanned 62 constructs (81% of constructs with at least one clone, 71% of constructs within the pool). For 65 constructs, one primer pair corresponding to one clone for each construct was received and used as a barcode and primer to isolate that clone. In total 65 primer pairs were received: 62 perfects, 3 known mutations. FIG. 10 illustrates that the amplification products of 64 of the 65 clones were cleanly detected (A1 missing, see FIG. 10).

### EQUIVALENTS

The present disclosure provides among other things novel methods and devices for high-fidelity gene assembly. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

Reference is made to U.S. Provisional Application No. 61/851,774 filed Mar. 13, 2013, U.S. Provisional Application No. 61/848,961 filed Jan. 16, 2013, U.S. Provisional Application No. 61/637,750 filed Apr. 24, 2012, U.S. Provisional Application No. 61/638,187 filed Apr. 25, 2012, and PCT International Application No. PCT/US2012/042597 filed Jun. 15, 2012. All publications, patents and sequence database entries mentioned herein are hereby incorporated by reference in their entirety as if each individual publication or patent or sequence database entry is specifically and individually indicated to be incorporated by reference.

1. A method for assembling a target nuclei acid, comprising:
(a) providing a plurality of nucleic acid molecules assembled from a pool of oligonucleotides;
(b) detecting a target nucleic acid that is misassembled, wherein the target nucleic acid is designed to be constructed from a subset of construction oligonucleotides out of the pool of oligonucleotides;
(c) selectively amplifying at least one of the subset of construction oligonucleotides; and
(d) assembling the amplified subset of construction oligonucleotides to form the target nucleic acid.

2. The method of claim 1, wherein the misassembled target nucleic acid (i) is underrepresented in the plurality of assembled nucleic acid molecules and/or (ii) contains an error.

3. The method of claim 2, wherein the error comprises missing one or more construction oligonucleotides.

4. The method of any one of claims 1-3, wherein the selectively amplifying step comprises polymerase based reaction using a first primer that is universal to all of the construction oligonucleotides in the subset and a second primer that is unique to the at least one construction oligonucleotide in the subset.

5. The method of claim 4, wherein each of the subset of construction oligonucleotides is designed to comprise a universal primer binding site located at a first end that binds the first primer, and a unique primer binding site located at a second end that binds the second primer.

6. The method of any one of claims 1-3 and 5, wherein in step (c), each of the subset of construction oligonucleotides is individually amplified.

7. The method of any one of claims 1-3 and 5, wherein in step (c), all of construction oligonucleotides in the subset are amplified in a single pool.

8. The method of any one of claims 1-3 and 5, wherein in step (d), the amplified subset of construction oligonucleotides are assembled via polymerase based assembly or ligase based ligation.

9. The method of any one of claims 1-3 and 5, wherein in step (d), the amplified subset of construction oligonucleotides are assembled hierarchically.

10. A method for assembling a target nucleic acid from a subset of construction oligonucleotides out of a pool of oligonucleotides, comprising:
(a) selectively, from the pool of oligonucleotides comprising the subset of construction oligonucleotides, and uniformly amplifying each of the subset of construction oligonucleotides; and
(b) assembling the amplified subset of construction oligonucleotides to form the target nucleic acid.

11. The method of claim 10, wherein the amplifying step comprises using a partial degenerate primer for each of the subset of construction oligonucleotides.

12. The method of claim 11, wherein in the partial degenerate primer, 1, 2, 3, 4, 5, 6, 7, 8, 9 or 10 positions each contain 2 or 3 or 4 possible nucleotides, and the remainder of the partial degenerate primer is specific to each construction oligonucleotide in the subset.

13. The method of claim 10 or 11, wherein the amplifying step comprises using a primer having a complement strand and an inhibitor strand, wherein the complement strand is partially complementary to each of the subset of construction oligonucleotides and the inhibitor strand is designed to compete with the corresponding construction oligonucleotide for binding to the complement strand.

14. The method of claim 13, wherein the complement strand is designed to have a complement region and a kick-off region, wherein the complement region binds the inhibitory strand and the kick-off region binds the corresponding construction oligonucleotide.

15. The method of claim 14, wherein the complement strand and the inhibitor strand are designed such that a first hybridization energy of the kick-off region and the corresponding construction oligonucleotide is substantially matched to a second hybridization energy of complement region and the inhibitor strand.
